(19) United States
(12) Patent Application Publication
(10) Pub. No.: US 2009/0170926 A1
(43) Pub. Date: Jul. 2, 2009

(54) Antiviral agents

(75) Inventors: Jonathan Alan Victor Coates, Beaumaris (AU); Eric Dale Jones, Bentleigh East (AU); Susan Cox, Beaumaris (AU); Ian Crosby, Hawthorn (AU); David Gerard Bourke, Nunawading (AU); Tyrone Pieter Jeynes, Pascoe Vale South (AU)

Correspondence Address: SEED INTELLECTUAL PROPERTY LAW GROUP PLLC, 701 FIFTH AVE, SUITE 5400, SEATTLE, WA, 98104, US

(73) Assignee: Monash Univeristy, Clayton (AU)

(21) Appl. No.: 10/594,847

(22) PCT Filed: Mar. 31, 2005
(86) PCT No.: PCT/AU2005/000453

§ 371 Date: Nov. 7, 2008

(30) Foreign Application Priority Data
Mar. 31, 2004 (AU) 2004901735

(51) Int. Cl. A61K 31/352; C07D 311/92; A61P 31/12

(52) U.S. Cl. 514/454; 549/389

(57) ABSTRACT

The present invention provides a method of treatment or prophylaxis of hepatitis B virus in a subject comprising administering an effective amount of a compound of formula (1) or a pharmaceutically acceptable derivative, salt or prodrug thereof. In addition, there is provided compounds of formula (1) and pharmaceutical compositions thereof. Further, methods of preparing compounds of formula (1) are disclosed.

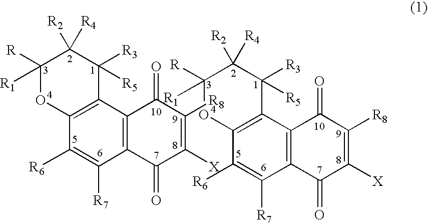

### FIELD OF THE INVENTION

The present invention relates to the use of naphthopyrans as agents in the treatment and/or prophylaxis of hepatitis B, pharmaceutical compositions for use in such therapy and novel naphthopyrans.

### BACKGROUND OF THE INVENTION

Infection with human hepatitis B virus is a major public health problem because of the ability of the virus to cause acute and chronic infections. Chronic hepatitis B virus infection (hereinafter referred to as “HBV”) causes serious liver disease in humans and frequently results in cirrhosis and hepatocellular carcinoma. Currently there is no completely effective therapy for the successful management of chronic HBV infections. The >250 million chronic HBV carriers throughout the world are unable to benefit from the commercial vaccine presently available.

Currently available therapies for HBV are only partially effective and may be accompanied by deleterious side effects. In addition, many patients develop antiviral resistance resulting in the loss of efficacy. Accordingly, a need exists for new effective treatments for HBV.

It has now been discovered that compounds of Formula (1) are active agents against hepatitis B virus.

### SUMMARY OF THE INVENTION

According to one aspect of the present invention there is provided a method of treatment or prophylaxis of hepatitis B virus in a subject comprising administering to said subject an effective amount of a compound of Formula (1):

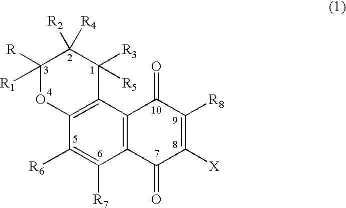

wherein X is OH, OR₉ or halo;

R and R₁ are independently selected from H, C₁₋₆ alkyl, C₂₋₆ alkenyl, C₂₋₆ alkynyl, C₃₋₆ cycloalkyl, aryl, or together with the carbon atom to which they are attached form a saturated or unsaturated C₃₋₆carbocyclic ring;

R₂ and R₃ are independently selected from H, C₁₋₆ alkyl, C₂₋₆ alkenyl, C₂₋₆ alkynyl, C₃₋₆ cycloalkyl or together with the bond between the carbon atoms to which they are attached form a double bond;

R₄ and R₅ are independently selected from H, C₁₋₆ alkyl, C₂₋₆ alkenyl, C₂₋₆ alkynyl, C₃₋₆ cycloalkyl, OH, OR₉, halo or NR₁₀R₁₀ or together with the bond between the carbon atoms to which they are attached form a double bond;

R₆ and R₇ are independently selected from H, C₁₋₆ alkyl, C₂₋₆ alkenyl, C₂₋₆ alkynyl, C₂₋₆ cycloalkyl, OH or OR₉;

R₈ is independently selected from H, C₁₋₆ alkyl, C₂₋₆ alkenyl, C₂₋₆ alkynyl, C₃₋₆ cycloalkyl, OH, OR₉ or halo;

R₉ is C₁₋₆ alkyl, C₂₋₆ alkenyl, C₂₋₆ alkynyl, C₃₋₆ cycloalkyl, aryl, C(═O)R₁₁ or S(O)₂R₁₂ or OR₉ is an amino acid residue;

each R₁₀ is independently selected from H and C₁₋₆ alkyl;

R₁₁ is C₁₋₂₁ alkyl, C₂₋₂₁ alkenyl, C₂₋₂₁ alkynyl, C₃₋₆ cycloalkyl, C₃₋₆ cycloalkyl C₁₋₆ alkyl, aryl or aryl C₁₋₆ alkyl; and

R₁₂ is C₁₋₆ alkyl, C₂₋₆ alkenyl, C₂₋₆ alkynyl or aryl.

According to a further aspect of the present invention there is provided a use of a compound of Formula (1) in the manufacture of a medicament for the treatment or prophylaxis of hepatitis B virus.

According to yet a further nspect of the present invention there is provided a method of treatment or prophylaxis of hepatitis B virus comprising administering an effective amount of a compound or Formula (1) and a second therapeutic agent.

According to another aspect of the invention there is provided a compound of Formula (1) with the proviso that when R and R₁ are both methyl and R₄ is OH or OR₉, R₅ is not selected from OH, OR₉ or NHR₁₀.

According to another aspect of the present invention there is provided a pharmaceutical composition comprising a compound of Formula (1) and a pharmaceutically acceptable carrier, excipient or adjuvant, with the proviso that in the compound of Formula (1) when R and R₁ are both methyl and R₄ is OH or OR₉, R₅ is not selected from OH, OR₉ or NHR₁₀.

According to the present invention the compounds of Formula (1) may be presented in the form of a pharmaceutically acceptable derivative, salt or prodrug.

### DETAILED DESCRIPTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference to any prior art in this specification is not, and should not be taken as, an acknowledgment or any form of suggestion that that prior art forms part of the common general knowledge in Australia.

As used herein, the term “halo” or “halogen” refers to fluorine (fluoro), chlorine (chloro), bromine (bromo) or iodine (iodo).

As used herein, the term “alkyl” either used alone or in compound terms such as NH(alkyl) or N(alkyl)₂, refers to monovalent straight chain or branched hydrocarbon groups, having 1 to 3, 1 to 6, 1 to 10 or 1 to 21 carbon atoms as appropriate. For example, suitable alkyl groups include, but are not limited to methyl, ethyl, propyl, isopropyl, n-butyl, sec-butyl, tert-butyl, pentyl, 2-methylbutyl, 3-methylbutyl, n-hexyl, 2-, 3- or 4-methylpentyl, 2-ethylbutyl, n-hexyl or 2-, 3-, 4- or 5-methylpentyl.

As used herein, the term “alkenyl” refers to straight chain or branched hydrocarbon groups having one or more double bonds hetween carbon atoms. Suitable alkenyl groups include, but are not limited to ethenyl, propenyl, isopropenyl, butenyl, pentenyl and hexenyl.

The term “alkynyl” as used herein, refers to straight chain or branched hydrocarbon groups containing one or more triple bonds. Suitable alkynyl groups include, but are not limited to ethynyl, propynyl, butynyl, pentynyl and hexenyl.

The term “cycloalkyl” as wed herein, refers to cyclic hydrocarbon groups. Suitable cycloalkyl groups include, but are not limited to cyclopropyl, cyclobutyl, cyclopentyl and cyclohexyl.

The term “aryl” as used herein, refers to C₆-C₁₀ aromatic hydrocarbon group, for example phenyl or naphthyl.

The term “heterocyclyl” when used alone or in compound words includes monocyclic, polycyclic, fused or conjugated hydrocarbon residues, preferably C₃₋₆, wherein one or more carbon atoms (and where appropriate, hydrogen atoms attached thereto) are replaced by a heteroulom so as to provide a non-aromatic residue. Suitable heteroatoms include, O, N and S. Where two or more carbon atoms are replaced, this may be by two or more of the same heteroatom or by different heteroatoms. Suitable examples of heterocyclic groups may include pyrrolidinyl, pyrrolinyl, piperidyl, piperazinyl, morpholino, indolinyl, imiazolidinyl, pyrazolidinyl, thiomorpholino, dioxanyl, tetrahydrofuranyl, tetrahydropyranyl, tetrahydropyrrolyl etc.

Each alkyl, alkenyl, alkynyl,cycloalkyl, aryl or heterocyclyl group may be optionally substituted with C₁₋₃ alkyl, OH, OC₁₋₃ alkyl, halo, CN, NO₂, CO₂H, CO₂C₁₋₃ alkyl, CONH₂, CONH(C₁₋₃ alkyl), CON(C₁₋₃ alkyl)₂, trifluoromethyl, NH₂, NH(alkyl) or N(alkyl)₂. For example, an optionally substituted aryl group may be a 4-methylphenyl or 4-hydroxyphenyl group, and an optionally substituted alkyl group may be 2-hydroxyethyl, trifluoromethyl or difluoromethyl.

As used herein, the term “amino acid residue” refers to an α-amino acid or a β-amino acid which is attached to the naphthopyrandione structure, preferably through the carboxylic acid group of the amino acid. The amino acid may be a L- or D-isomer and may have a naturally occurring side chain or a non-naturally occurring side chain. The amino acid may also be further substituted in the α-position or the β-position with a group selected from —C₁-C₆ alkyl, —C₂-C₆ alkenyl, —C₂-C₆ alkynyl, —(CH₂)ₙCOR₃, —(CH₂)ₙRₕ, —PO₃H, —(CH₂)ₙheterocyclyl or —(CH₂)ₙaryl where Rₙ is —OH, —NH₂, —NHC₁-C₃ alkyl, —OC₁-C₃ alkyl or —C₁-C₃ alkyl and R_{b} is —OH, —SH, —SC₁-C₃ alkyl, —OC₁-C₃ alkyl, —C₃-C₆ cycloalkyl, —C₃-C₆ cycloalkenyl, —NH₂, —NHC₁-C₃ alkyl or —NHC(C═NH)N H₂, n is 0 or an integer from 1 to 6 and where each alkyl, alkenyl, alkynyl, cycloalkyl, cycloalkenyl, aryl or heterocyclyl group may be substituted with one or more groups selected from —OH, —NH₂, —NHC₁-C₃ alkyl, —OC₁-C₃ alkyl, —SH, —SC₁-C₃ alkyl, —CO₂H, —CO₂C₁-C₃ alkyl, —CONH₂ or —CONHC₁-C₂ alkyl.

The term “α-amino acid” as used herein, refers to a compound having an amino group and a carboxyl group in which the amino group and the carboxyl group are separated by a single carbon atom, the α-carbon atom. An α-amino acid includes naturally occurring and non-naturally occurring L-amino acids and their D-isomers and derivatives thereof such as salts or derivatives where functional groups are protected by suitable protecting groups. The α-amino acid may also be further substituted in the α-position with a group selected from —C₁-C₁₀ alkyl, —C₂-C₁₀ alkenyl, —C₂-C₁₀ alkynyl, —(CH₂)ₙCORₐ, —(CH₂)ₙR_{b}, —PO₃H, —(CH₂)ₙ heterocyclyl or —(CH₂)ₙ aryl where Rₙ is —OH, —NH₂, —NHC₁-C₃ alkyl, —OC₁-C₃ alkyl or C₁-C₃ alkyl and R_{b} is —OH, —SH, —SC₁-C₃ alkyl, —OC₁-C₃ alkyl, —C₃-C₁₂ cycloalkyl, —C₃-C₁₂ cycloalkenyl, —NH₂, —NHC₁-C₃ alkyl or —NHC(C═NH)NH₂, n is 0 or an integer from 1 to 10 and where each alkyl, alkenyl, alkynyl, cycloalkyl, cycloalkenyl, aryl or heterocyclyl group may be substituted with one or more groups selected from —OH, —NH₂, —NHC₁-C₃ alkyl, —OC₁-C₃ alkyl, —SH, —SC₁-C₃ alkyl, —CO₂H, —CO₂C₁-C₃ alkyl, —CONH₂ or —CONHC₁-C₃ alkyl.

As used herein, the term “β-amino acid” refers to an amino acid that differs from an α-amino acid in that there are two (2) carbon atoms separating the carboxyl terminus and the amino terminus. As such, β-amino acids with a specific side chain can exist as the R or S enantiomers at either of the α (C2) carbon or the β (C3) carbon, resulting in a total of 4 possible isomers for any given side chain. The side chains may be the same as those of naturally occurring α-amino acids or may be the side chains of non-naturally occurring amino acids.

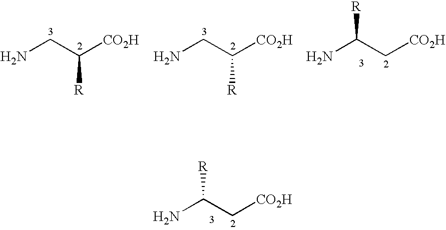

Furthermore, the β-amino acids may have mono-, di-, tri- or tetra-substitution at the C2and C3 carbon atoms. Mono-substitution may be at the C2 or C3 carbon atom. Di-substitulion includes two substitucnts at the C2 carbon atom, two substitucnts at the C3carbon atom or one substituent at each of the C2 and C3 carbon atoms. Tri-substitution includes two substituents at the C2 carbon atom and one substituent at the C3 carbon atom or two substituents at the C3 carbon atom and one substituent at the C2 carbon atom. Tetra-substitution provides for two substituents at the C2 carbon atom and two substituents at the C3 carbon atom. Suitable substituents include —C₁-C₆ alkyl, —C₂-C₆ alkenyl, —C₂-C₆ alkynyl, —(CH₂)ₙCORₐ, —(CH₂)ₙR_{b}, —PO₃H, —(CH₂)ₙ heterocyclyl or —(CH₂)ₙ aryl where Rₙ is —OH, —NH₂, —NHC₁-C₃ alkyl, —OC₁-C₃ alkyl or —C₁-C₃ alkyl and R_{b} is —OH, —SH, —SC₁-C₃ alkyl, —OC₁-C₃ alkyl, —C₃-C₆ cycloalkyl, —C₃-C₆ cycloalkenyl, —NH₂, —NHC₁-C₃ alkyl or —NHC(C═NH)NH₂, n is 0 or an integer from 1 to 6 and where each alkyl, alkenyl, alkynyl, cycloalkyl, cycloalkenyl, aryl or heterocyclyl group may be substituted with one or more groups selected from —OH, —NH₂, —NHC₁-C₃ alkyl, —OC₁-C₃ alkyl, —SH, —SC₁-C₃ alkyl, —CO₂H, —CO₂C₁-C₃ alkyl, —CONH₂ or —CONHC₁-C₃ alkyl.

The term “non-naturally occurring amino acid” as used herein, refers to amino acids having a side chain that does not occur in the naturally occurring L-α-amino acids. Examples of non-natural amino acids and derivatives include, but are not limited to, use of norleucine, 4-amino butyric acid, 4-amino-3-hydroxy-5-phenylpentanoic acid, 6-aminohexanoic acid, t-butylglcine, norvaline, phenylglycine, ornithine, sarcosine, 4-amino-3-hydroxy-6-methylheptanoic acid, 2-thienyl alanine and/or D-isomors of amino acids.

It will also be recognised that the compounds of formula (1) may possess asymmetric centres and are therefore capable of existing in more than one stereoisomeric form. The invention thus also relates to compound in substantially pure isomeric form at one or more asymmetric centres eg., greater than about 90% ee, such as about 95% or 97% ee or greater than 99% ee, as well as mixtures, including racemic mixtures, thereof. Such isomers may be prepared by asymmetric synthesis, for example using chiral intermediates, or by chiral resolution.

The term “pharmaceutically acceptable derivative” may include any pharmaceutically acceptable salt, hydrate or prodrug, or any other compound which upon administration to a subject, is capable of providing (directly or indirectly) a compound of formula (1) or an antivirally active metabolite or residue thereof.

Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, bcnzenesulphonic, salicylic, sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, zinc, ammonium, alkylammonium such as salts formed from triethylamine, alkoxyammonium such as those formed with ethanolamine and salts formed from ethylenediamine, choline or amino acids such as arginine, lysine or histidine.

Basic nitrogen-containing groups may be quarternised with such agents as lower alkenyl halide, such as methyl, ethyl, propyl, and butyl chlorides, bromides and iodides; dialkyl sulfates like dimethyl and diethyl sulfate; and others.

The term “prodrug” is used in its broadest sense and encompasses those derivatives that are converted in vivo to the compounds of the invention. Such derivatives would readily occur to those skilled in the art, and include, for example, compounds in which a free hydroxy group is converted into a group, such as an ester, carbonate or carbamate, which is capable of being converted in vivo back to a hydroxy group. A prodrug may include modifications of one or more of the functional groups of a compound of formula (1). For example, similar to the approach described in U.S. Pat. No. 5,672,607, antiviral naphthopyran prodrugs having enhanced water-solubility (e.g., which are better for parenterally administered compositions) may be prepared by chemical reduction of the quinone functionalities to the corresponding quinols, followed by reaction with phosphorous oxychloride to give the corresponding phosphoric acid esters. After in vivo administration of a composition containing such a solubilized antiviral naphtopyran prodrug, the prodrug will be readily hydrolysed to the corresponding quinol, which thereafter will oxidize to re-form in vivo the active parent antiviral naphthopyrandione. Likewise, other kinds of derivatives may be prepared from the reduced quinol derivatives of the antiviral naphthopyrandione; these can also serve as prodrugs for use in therapeutic compositions. For example, other types of esterification (e.g., acetylation) may be used to produce antiviral naphthopyran prodrugs, such as for example 7,8,10-triacetoxy-3,3-dimethyl-3H-naptho[2,1-b]pyran. Again, after in vivo administration the prodrug would be readily hydrolysed and oxidized to its parent active antiviral naphthopyran compound.

In a first aspect, there is provided a method of treatment or prophylaxis of hepatitis B virus in a subject comprising administering to said subject an effective amount of a compound of Formula (1):

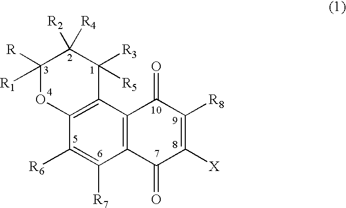

wherein X is OH, OR₉ or halo;

R and R₁ are independently selected from H, C₁₋₆ alkyl, C₂₋₆ alkenyl, C₂₋₆ alkynyl, C₃₋₆ cycloalkyl, aryl, or together with the carbon atom to which they are attached form a saturated or unsaturated C₃₋₆ carbocyclic ring;

R₂ and R₃ are independently selected from H, C₁₋₆ alkyl, C₂₋₆ alkenyl, C₂₋₆ alkynyl, C₃₋₆ cycloalkyl or together with the bond between the carbon atoms to which they are attached form a double bond;

R₄ and R₅ are independently selected from H, C₁₋₆ alkyl, C₂₋₆ alkenyl, C₂₋₆ alkynyl, C₃₋₆ cycloalkyl, OH, OR₉, halo or NR₁₀R₁₀ or together with the bond between the carbon atoms to which they are attached form a double bond;

R₆ and R₇ are independently selected from H, C₁₋₆ alkyl, C₂₋₆ alkenyl, C₂₋₆ alkynyl, C₃₋₆ cycloalkyl, OH or OR₉;

R₈ is independently selected from H, C₁₋₆ alkyl, C₂₋₆ alkenyl, C₂₋₆ alkynyl, C₃₋₆ cycloalkyl, OH, OR₉ or halo;

R₉ is C₁₋₆ alkyl, C₂₋₆ alkenyl, C₂₋₆ alkynyl, C₃₋₆ cycloalkyl, aryl, C(═O)R₁₁ or S(O)₂R₁₂ or OR₉ is an amino acid residue;

each R₁₀ is independently selected from H and C₁₋₆ alkyl;

R₁₁ is C₁₋₂₁ alkyl, C₂₋₂₁ alkenyl, C₂₋₂₁ alkynyl, C₃₋₆ cycloalkyl, C₃₋₆ cycloalkyl C₁₋₆ alkyl, aryl or aryl C₁₋₆ alkyl; and

R₁₂ is C₁₋₆ alkyl, C₂₋₆ alkenyl, C₂₋₆ alkynyl or aryl.

In another aspect there is provided a compound of Formula (1), with the proviso that when R and R₁ are both methyl and R is OH or OR₉, R₅ is not selected from OH, OR₉ or NHR₉.

In a preferred embodiment one or move of the following definitions apply;

X is OH, OC₁₋₆ alkyl or halo;

R and R₁ are independcndy selected from H or C₁₋₃ alkyl or together with the carbon atom to which they are attached form a saturated or unsaturated C₃₋₆ carbocyclic ring;

R₂ and R₃ are each hydrogen;

R₄ and R₅ are independently selected from H, OH, OR₉, or halo or together with the bond between the carbon atoms to which they are attached form a double bond;

R₆ and R₇ are independently selected from H, OH, C₁₋₆ alkyl, C₁₋₆ alkoxy;

R₈ is H, OH, OR₉, C₁₋₆ alkyl or halo;

R₉ is C(═O)R₁₁ or S(O)₂R₁₂;

R₁₁ is C₁₋₂₁ alkyl;

R₁₂ is C₁₋₆ alkyl, phenyl or tosyl;

Preferred compounds of the invention include those of formula (2);

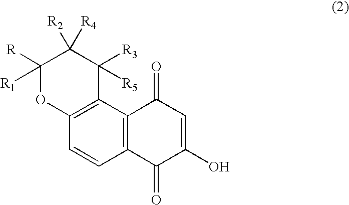

wherein R, R₁, R₂, R₃, R4 and R5 are defined as for formula (1).

Preferred compounds of the invention include:
8-hydroxy-3,3-dimethyl-3H-naphtho[2,1-b]pyran-7,10-dione,
8-hydroxy-3,3-dimethyl-1,2-dihydro-3H-naphtho[2,1-b]pyran-7,10-dione,
9-bromo-8-hydroxy-3,3-dimethyl-1,2-dihydro-3H-naphtho[2,1-b]pyran-7,10-dione,
9-bromo-8-hydroxy-3,3-dimethyl-3H-naphtho[2,1-b]pyran-7,10-dione,
9-bromo-3,3-dimethyl-8-(4-methylbenzenesulfonyloxy)-1,2-dihydro-3H-naphtho[2,1-b]pyran-7,10-dione,
9-bromo-3,3-dimethyl-8-(4-methylbenzenesulfonyloxy)-3H-naphtho[2,1-b]pyran-7,10-dione,
8-acetoxy-3,3-dimethyl-3H-naphtho[2,1-b]pyran-7,10-dione,
2,9-dibromo-1,8-dihydroxy-3,3-dimethyl-1,2-dihydro-3H-naphtho[2,1-b]pyran-7,10-dione,
8,9-dichloro-3,3-dimethyl-1,2-dihydro-3H-naphtho[2,1-b]pyran-7,10-dione,
7,8,10-triacetoxy-3,3-dimethyl-3H-naphtho[2,1-b]pyran,
9-Bromo-8-hydroxy-3,3-dimethyl-3H-naphtho[2,1-b]pyran-7,10-dione.
9-Bromo-8-hydroxy-3,3-dimethyl-3H-naphtho[2,1-b]pyran-7,10-dione.
9-Bromo-3,3-dimethyl-8-(4-methylbenzenesulfonyloxy)-1,2-dihydro-3H-naphtho[2,1-b]pyran-7,10-dione.
9-Bromo-3,3-dimethyl-8-(4-methylbenzenesulfonyloxy)-3H-naphtho[2,1-b]pyran-7,10-dione,
8-Bromo-3,3-dimethyl-9-(4-methylbenzenesulfonyloxy)-3H-naphtho[2,1-b]pyran-7,10-dione,
8-Bromo-3,3-dimethyl-9-(4-methylbenzenesulfonyloxy)-1,2-dihydro-3H-naphtho[2,1-b]pyran-7,10-dione.
8,9-Dichloro-3,3-dimethyl -1,2-dihydro-3H-naphtho[2,1-b]pyran-7,10-dione,
Sodium 3,3-dimethyl-7,10-dioxo-7,10-dihydro-3H-benzo[f]chromen-8-olate;
Sodium 3,3-dimethyl-7,8-dioxo-7,8-dihydro-3H-benzo[f]chromen-10-olate
8-Hydroxy-3-methyl-3-phenyl-3H-benzo[f]chromene-7,10-dione, and
8-Hydroxy-3,3-diphenyl-3H-benzo[f]chromene-7,10-dione,

Preferably the compound of Formula (1) is:
8-hydroxy-3,3-dimethyl-3H-naphtho[2,1-b]pyran-7,10-dione (compound (1)),
8-hydroxy-3,3-dimethyl-1,2-dihydro-3H-naphtho[2,1-b]pyran-7,10-dione (compound (2)).

In another embodiment the compounds of the invention include those of formula (3):

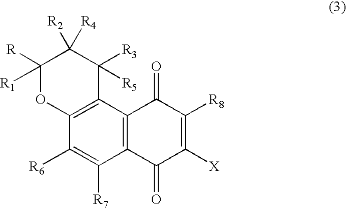

wherein R, R₁, R₂, R₃, R₅, R₆, R₇, R₈, and X are as defined for formula (1) and R₄ is selected from H, C₁₋₆ alkyl, C₂₋₆ alkenyl, C₂₋₆ alkynyl, C₃₋₆ cycloalkyl, halo or NR₁₀R₁₀ or together with R₅ and the bond between the carbon atoms to which R₄ and R₅ are attached, form a double bond.

Compounds of Formula (1) may be prepared using the methods depicted or described herein or known in the art. It will be understood that minor modifications to methods described herein or known in the art may be required to synthesise particular compounds of Formula (1). General synthetic procedures applicable to the synthesis of compounds may be found in standard references such as Comprehensive Organic Transformations, R. C. Lorock, 1989, VCH Publishers and Advanced Organic Chemistry, J. March, 4th Edition (1992), Wiley InterScience, and references therein. It will also be recognised that certain reactive groups may require protection and deprotection during the synthetic process. Suitable protecting and deprotecting methods for reactive functional groups are known in the art for example in Protective Groups in Organic Synthesis, T. W. Greene & P. Wutz, John Wiley & Son, 3rd Edition, 1999.

The compounds of the present invention may be prepared according to the general procedure of Scheme 1.

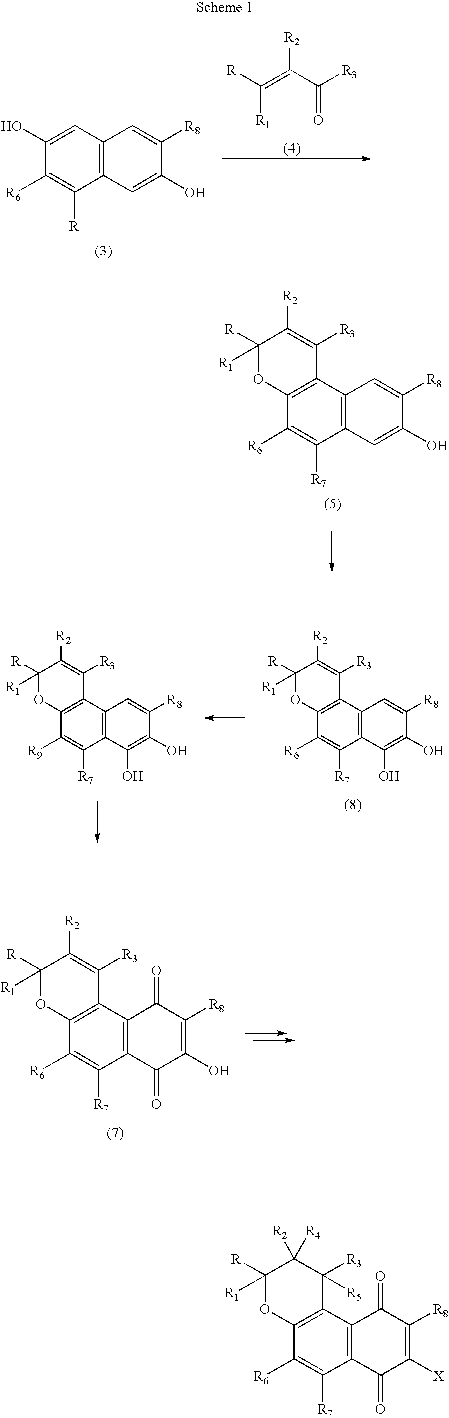

An appropriately substituted 2,6-dihydroxynaphthalene (3) is reacted with an appropriately substituted enal or enone (4) in the presence of a suitable base to effect cyclisation and provide a naphthopyranol (5). The naphthopyranol is then oxidised by a suitable oxidant to the corresponding intermediate orthoquinone (6), before being reduced by a suitable reducing agent and further oxidised by a suitable oxidant to tho desired naphthopyrandione (7). Further modification of the substituents on the naphthopyrandione may be effected using chemical approaches known to those skilled in the art for the generation of the desired substituent or substituents. Those skilled in the art may utilise conventional approaches to protect and deprotect certain functional groups during the reaction sequence. Such methods are well known in the art and include for example those described by Greene and Wutz (supra). The reaction sequence described in Examples 1 and 2 exemplify the preparation of compounds (1) and (2) and provide an example of how the reaction sequence of Scheme 1 is utilised. Those skilled in the art will appreciate that a wide variety of reaction conditions, including solvents, bases, oxidising agents, reducing agents, temperature and time of the reaction, may be utilised to effect the desired transformation.

A substituted enone such as (4) may, dependant upon the exact nature of the reagents and conditions used, add to the substituted 2,6-dihydroxynaphthalene (3) in the opposite orientation to that shown in Scheme 1 and still provide a naphthopyran product. Such a reaction is shown in Scheme 2, and provides naphthopyranol (8). Naphthopyranol (8) may be isomerised to provide a naphthopyranol effectively of general formula (5), which may then be subject to further reaction in accordance with the general procedures of Scheme 1 to provide compounds of Formula (1).

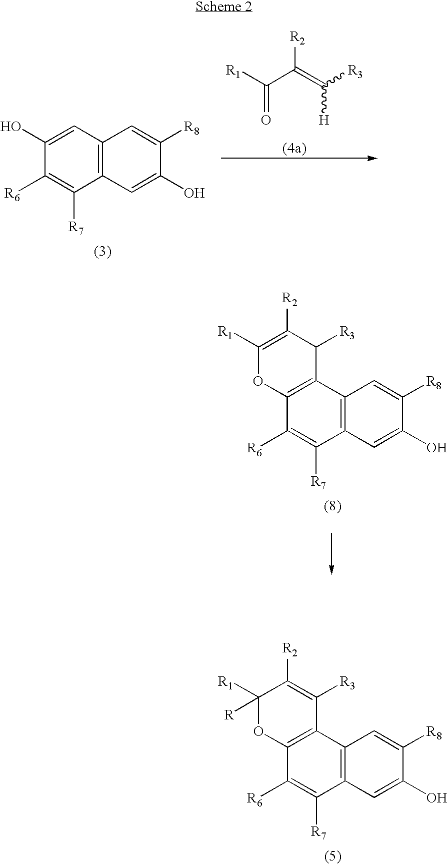

Alternative synthetic procedures which provide compounds of Formula (1) are shown in Schemes 3 and 4. In Scheme (3) an appropriately substituted butyne (9) is reacted with an appropriately substituted hydroxy tetralone (10). The group L is any suitable leaving group and includes groups such as a bromo, chloro, and hydroxyl. Reaction between the tetralone and the butyne may be acid or base catalysed to provide naphthopyran (12). In some cases the reaction may be conducted in one pot however an intermediate (11) may be isolated. Intermediate (11) may conveniently be cyclised for example by heating in the presence of a suitable base, such as diethylaniline. The cyclised product (12) is then oxidised to afford the quinone (13) which may then be further modified to provide other compounds of Formula (1).

Scheme (4) outlines a similar reaction sequence to that of Scheme (3) which would start with an appropriately substituted hydroxy naphthalene. This is based upon work reported by Bigi et al., J. Org. Chem., 62, 7024-7027 (1997). The cyclised naphthopyran (15) could be treated as per compound (5) of Scheme (1) to provide compounds of the invention.

Many other methods of preparing benzopyrans have been reported in the chemical literature and those skilled in the art may adapt these methods to provide compounds of the present invention, sec for example, Ishino et al., Syn. Comm., 31, 439-448 (2001).

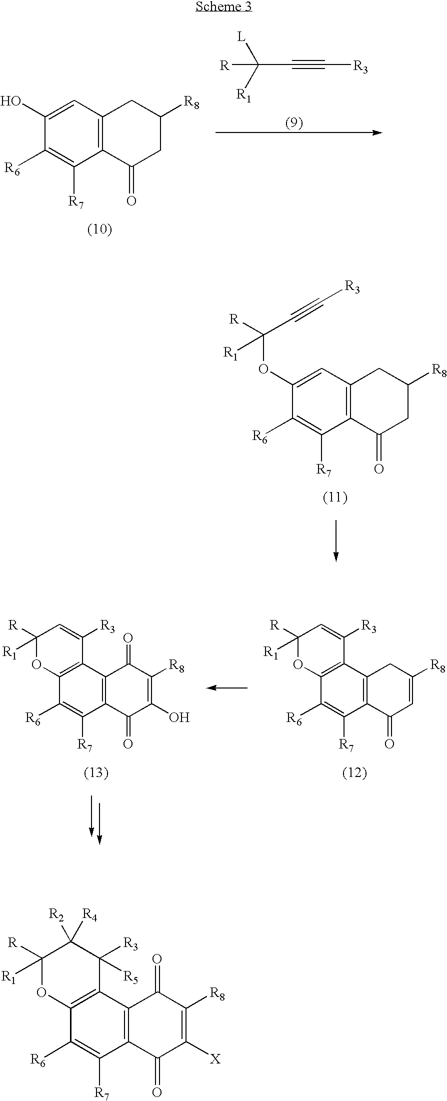

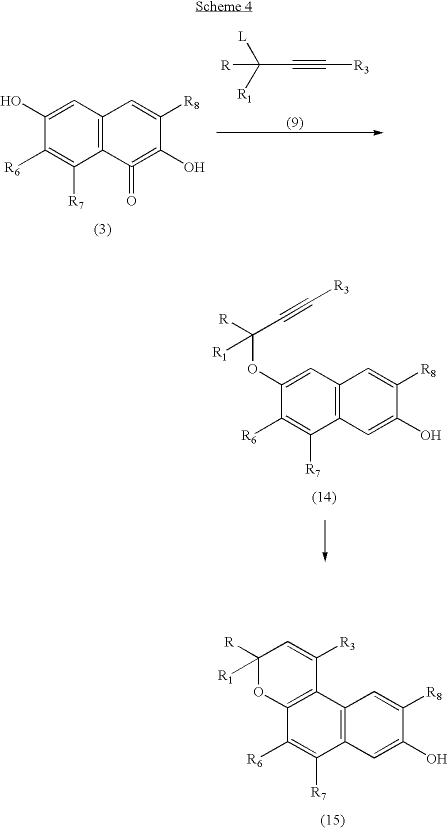

Further modification may include derivatisation of double bonds. For example, when R₄ and R₅ together with the bond between the carbon atoms to which they are attached form a double bond, the double bond may be derivatised by addition, oxidation or reduction reactions. An example of possible derivatisation of such a double bond is given in Scheme 5. Following reductive acetylation to protect the quinone portion of the compound, epoxidation of the pyran double bond, subsequent ring opening of the epoxide with an amine, and deprotection and oxidation to regenerate the quinone may be effected. Those skilled in the art could readily determine appropriate reagents and conditions to effect such transformations.

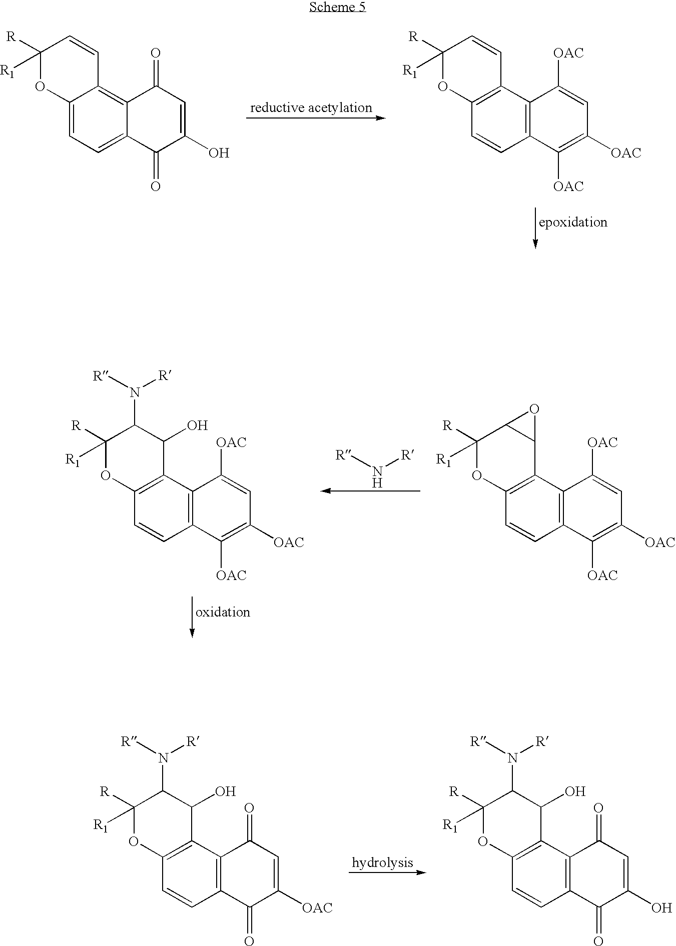

A person skilled in the art would be able to modify such a reaction scheme by using different reagents to open the epoxide, using asymmetric epoxidation catalysts and varying the nature of the substituents.

As used herein, the term “effective amount” relates to an amount of compound which, when administered according to a desired dosing regimen, provides the desired hepatitis B virus treatment or therapeutic activity, or disease prevention. Dosing may occur at intervals of minutes, hours, days, weeks, months or years or continuously over any one of these periods. A therapeutic, or treatment, effective amount is an amount of the compound which, when administered according to a desired dosing regimen, is sufficient to at least partially attain the desired therapeutic effect, or delay the onset or, or inhibit the progression of or halt or partially or fully reverse the onset or progression of hepatitis B virus. A prevention effective amount is an amount of compound which whea administered according to the desired dosing regimen is sufficient to at least partially prevent or delay the onset of a particular disease or condition.

Yet another aspect of the present invention provides a use of a compound of Formula (1) in the preparation of a medicament for treating or preventing hepatitis B virus.

Suitable dosages may lie within the range of about 0.1 ng per kg of body weight to 1 g per kg of body weight per dosage. The dosage is preferably in the range of 1 μg to 1 g per kg of body weight per dosage, such as is in the range of 1 mg to 1 g per kg of body weight per dosage. In one embodiment, the dosage is in the range of 1 mg to 500 mg per kg of body weight per dosage. In another embodiment, the dosage is in the range of 1 mg to 250 mg per kg of body weight per dosage. In yet another preferred embodiment, the dosage is in the range of 1 mg to 100 mg per kg of body weight per dosage, such as up to 50 mg per kg of body weight per dosage. In yet another embodiment, the dosage is in the range of 1 μg to 1 mg per kg of body weight per dosage.

Suitable dosage amounts and dosing regimens can be determined by the attending physician and may depend on the severity of the condition as well as the general age, health and weight or the subject.

The active ingredient may be administered in a single dose or a series of doses. While it is possible for the active ingredient to be administered alone, it is preferable to present it as a composition, preferably as a pharmaceutical composition.

According to a further embodiment there is provided a method of treatment or prophylaxis of hepatitis B virus comprising administering an effective amount of a compound of Formula (1) and a second therapeutic agent.

When administered as a combination, the compound of Formula (1) and the second therapeutic agent may be administered simultaneously, separately or sequentially.

The second therapeutic agent may be a known antiviral or antiretrovital agent or another pharmaceutical used in the treatment of viral infections. Representative examples of suitable second therapeutic agents include immunomodulators, immunostimulants and antibiotics. Exemplary antiviral agents include acyclovir, val-acyclovir, penciclovir, famciclovir, ganciclovir, foscarnet, ribavirin, interferon-alpha, PEG-interferon-alpha, lamivudine, adefovir, thymosin alpha 1, entecavir, telbivudine, emtricitabine, elvucitabine, MCC-478, hepavir B, MIV-210, valtorcitabine, HepeX-B, Zidovudine, didanosine, zalcitabine, stavudine, lamivudine, abacavir, tenofovir, emtricitabine, saquinavir, indinavir, nelfinavir, amprenavir, ritonavir, azatanavir, nevirapine, delavirdine, efavirenz, enfurvitide, trizivir, combivir, kaletra, MIV310, mozenavir, SPD754, SPD746, T1249, TMC125, TMC114, VX-175, tipranavir other non-nucleoside reverse transcriptase inhibitors and protease inhibitors. Exemplary immunomodulators and immunoslimulants include interferon alpha, PEG-interferon, thymosin alpha 1, HepeX-B, HBV immunoglobulin, HBV monoclonal antibodies, and vaccines such as EngerixB, Havrix, H-B-Vax II, infanrix hep B, twinrix. Preferably the second therapeutic agent is an agent suitable for the treatment or prophylaxis of hepatitis B virus in n subject. Such therapeutic agents include, but are not limited to interferon-alpha, PEG-interferon-alpha, lamivudine, adefovir, thymosin alpha 1, entecavir, telbivudine, emtricitabine, elvucitabine, MCC-478, hepavir B, MIV-210, valtorcitabine, and HepeX-B.

Still another aspect of the present invention relates to a pharmaceutical composition comprising a compound of Formula (1) and a pharmaceutically acceptable carrier, diluent or excipient.

The formulation of such compositions is well known to those skilled in the art. The composition may contain pharmaceutically acceptable additives such as carriers, diluents or exeipients. These include, where appropriate, all conventional solvents, dispersion agents, fillers, solid carriers, coating agents, antifungal and antibacterial agents, dermal penetration agents, surfactants, isotonic and absorption agents and the like. It will be understood that the compositions of the invention may also include other supplementary physiologically active agents.

The carrier must be pharmaceutically acceptable in the sense of being compatible with the other ingredients of the composition and not injurious to the subject. Compositions include those suitable for oral, rectal, inhalational, nasal, transdermal, topical (including buccal and sublingual), vaginal or parenteral (including subcutaneous, intramuscular, intraspinal, intravenous and intradermal) administration. The compositions may conveniently be presented in unit dosage form and may be prepared by any methods well known in the art of pharmacy. Such methods include the step of bringing into association the active ingredient with the carrier which constitutes one or more accessory ingredients. In general, the compositions are prepared by uniformly and intimately bringing into association the active ingredient with liquid carriers or finely divided solid carriers or both, and then if necessary shaping the product.

Depending on the disease or condition to be treated, it may or may not be desirable for a compouad of Formula (1) to cross the blood/brain barrier. Thus the compositions for use in the present invention may be formulated to be water or lipid soluble.

Compositions of the present invention suitable for oral administration may be presented as discrete units such as capsules, sachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (eg inert diluent, preservative, disintegrant (eg. sodium starch glycolate, cross-linked polyvinyl pyrrolidone, cross-linked sodium carboxymethyl cellulose)) surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Compositions suitable for topical administration in the mouth include lozenges comprising the active ingredient in a flavoured base, usually sucrose and acacia or tragacanth gum; pastilles comprising the active ingredient in an inert basis such as gelatin and glycerin, or sucrose and acacia gum; and mouthwashes comprising the active ingredient in a suitable liquid carrier.

The compounds of Formula (1) may also be administered intranasally or via inhalation, for example by atomiser, aerosol or nebulizer means.

Compositions suitable for topical administration to the skin may comprise the compounds dissolved or suspended in any suitable carrier or base and may be in the form of lotions, gel, creams, pastes, ointments and the like. Suitable carriers include mineral oil, propylene glycol, polyoxyethylene, polyoxypropylene, emulsifying wax, sorbitan monostearate, polysorbate 60, cetyl esters wax, cetearyl alcohol, 2-octyldodecanol, benzyl alcohol and water. Transdermal devices, such as patches, may also be used to administer the compounds of the invention.

Compositions for rectal administration may be presented as a suppository with a suitable carrier base comprising, for example, cocoa butter, gelatin, glycerin or polyethylene glycol.

Compositions suitable for vaginal administration may be presented as pessaries, tampons, creams, gels, pastes, foams or spray formulations containing in addition to the active ingredient such carriers as are known in the art to be appropriate.

Compositions suitable for parenteral administration include aqueous and non-aqueous isotonic sterile injection solutions which may contain anti-oxidants, buffers, bactericides and solutes which render the composition isotonic with the blood of the intended recipient; and aqueous and non-aqueous sterile suspensions which may include suspending agents and thickening agents. The compositions may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepured from sterile powders, granules and tablets of the kind previously described.

Preferred unit dosage compositions are those containing a daily dose or unit, daily sub-dose, as herein above described, or an appropriate fraction thereof, of the active ingredient.

It should be understood that in addition to the active ingredients particularly mentioned above, the compositions of this invention may include other agents conventional in the art having regard to the type of composition in question, for example, those suitable for oral administration muy include such further agents as binders, sweeteners, thickeners, flavouring agents, disintegrating agents, coating agents, preservatives, lubricants and/or time delay agents. Suitable sweeteners include sucrose, lactose, glucose, aspartame or saccharine. Suitable disintegrating agents include corn starch, methylcellulose, polyvinylpyrrolidone, xanthan gum, bentonite, alginic acid or agar. Suitublc flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

The invention will now be described with reference to the following examples which are included for the purpose of illustration only and are not intended to limit the generality of die invention hereinbefore described.

### EXAMPLES

### Example 1

Compound 1: 9-Hydroxy-3,3dimethyl-3H-naphtho[2,1-b]pyran-7,10-dione
Step 1: 3,3-Dimethyl-3H-naphtho[2,1-b]pyran-8-ol

A mixture of 2,6-dihydroxynaphthalene (50.0 g, 0.312 mol), 3-methyl-2-butenal (30 mL, 26.24 g, 0.312 mol) and pyridine (38 mL, 37.02 g, 0.468 mol) were heated under reflux for 3.5 h. The mixture was cooled to room temperature, diluted with dichloromethane (500 mL), filtered through a sintered glass runnel (porosity 3) then washed with aqueous hydrochloric acid solution (1 M, 2×250 mL) and water (1×250 mL). The organic layer was exracted with a solution of aqueous sodium hydroxide (2 M, 1×250 mL and 1×125 mL) and the combined aqueous extracts cooled in an ice-salt bath, acidified (with stirring) with aqueous hydrochloric acid solution (5 M) until a creamy-white precipitate formed (pH ˜2). The solid was stirred for an additional 10 min with cooling, collected by filtration, washed with water and dried under high vacuum at 40° C. to afford the desired crude product as a fluffy white-grey solid (43.9 g, 62%). The crude product was used in the subsequent reaction without further purification.

Recrystallised from diethyl ether/hexane m.p. 120-123°. δ (¹H) (300 MHz, CDCl₃)1.47, s; 2×CH₃; 4.77, s, OH; 5.71, d, J 10.2 Hz, H2; 6.97, d, J 10.2 Hz, H1; 7.02, d, J 8.7 Hz, H5; 7.08, s, H7; 7.10. dd, J 8.7. 2.7 Hz, H9; 7.48, d, J 8.7 Hz, H6; 7.85, d, J 8.7 Hz, H10. m/z (ES⁻, 100 V) 471 (2M+H+H₂O, 100%), 245 (M+H+H₂O, 62), 227 (M+H, 63).

Step 2: 3,3-Dimethyl-3H-naphtho[2,1-b]pyran-7,8-dione

To an oxygen saturated solution of 3,3-dimethyl-3H-naphto-[2,1-b]pyran-8-ol (3.0 g, 13 mmol) in acetonilrile (70 mL) was added catalytic amounts of N,N-Bis(salicylidene)ethylenediamineocobalt(II) hydrate, ([Co(II)(Salen)₂]) (300 mg, 0.91 mmol, 7 mol %), and oxygen was bubbled through the mixture until the reaction was deemed completed (generally 4.5 h) by TLC (hexane-ethyl acetate 4:) or HPLC. The orange/brown reaction mixture was diluted with ethyl acetate and the entire mixture filtered through a plug of flash silica (11×7 cm) to remove the catalyst. The plug was washed with ethyl acetate until the eluent was nearly colourless. The solvent concentrated in vacuo and the residue dried under high vacuum to afford the desired crude product as an orange solid (2.73 g, 86%). The crude product was used in the subsequent reaction without further purification.

The product was recrystallised from ethyl acetate/hexanes to afford red needles; m.p. 189-193° δ (¹H) (300 MHz, CDCl₃) 1.50, s, 2×CH₃; 5.92, d, J 10.4 Hz, H2; 6.43, d, J 10.4 Hz, H1; 6.71, d, J 10.5 Hz, H9; 6.84, d, J 8.6 Hz, H5; 7.72, d, J 10.5 Hz, H10; 7.97, d, J 8,6 Hz, H6. m/z (ES⁺, 30 V) 263 (M+Na, 9%), 242 (M+H+1, 19), 241 (M+H, 100).

Step 3: 9-Hydroxy-3,3-dimethyl-3H-naphtho[2,1-b]pyran-7,10-dione

A solution of 3,3-dimethyl-3H-naphtho[2,1-b]pyran-7,8-dione (4.59 g, 19.1 mmol) in toluene (340 mL) was washed twice with a solution of sodiuun dithionite (24.9 g, 0.143 mol) in water (250 mL). The yellow organic layer was then added in one portion to a oxygen saturated solution of potassium tert-butoxide (12.19 g, 115 mmol) in tert-butanol (110 mL) and the resulting mixture was stirred at room temperature with oxygen bubbling for an additional 30 min (NOTE: longer periods appears to result in reduced yield). The resultant dark red solution was acidified with aqueous hydrochloric acid solution (initially 2 M then 5 M) until the colour turns yellow/orange (pH˜1), then water (˜40 mL) was added to dissolve the formed salt, and the layers separated. The organic phase was washed with water (1×85 mL), and then extracted with a saturated aqueous sodium hydrogen carbonate solution (5×85 mL). The combined aqueous extracts were transferred back into the separating funnel and allowed to settle for 1 h (to separate further amounts of toluene) and the layers separated again. The combined base extracts were cooled in an ice-salt bath, carefully acidified (aqueous hydrochloric acid, 5M, ˜80 mL) dropwisc over 30 min with stirring until the colour turns pale yellowish (pH˜1-2). The resultant precipitate was further cooled in the ice-salt bath with stirring, the solid collected, washed with water (˜100 mL) to remove coloured impurities, and the orange/brown solid was recrystallised (absolute ethanol) to afford the desired product as orange coloured crystals (1.04 g, 21%), m.p. 208° δ (¹H) (300 MHz, CDCl₃)1.48, s, 2×CH₃, 5.94, d, J 10.5 Hz, H2; 6.23, s, H9; 7.03, d, J 8.4 Hz, H5; 7.83, d, J 10.5 Hz, H1; 7.99, d, J 8.4 Hz, H6, m/z (ES⁺, 100 V) 279 (M+Na, 100%), 257 (M+H, 46), 159 (46), 137 (49), 86 (44), 59 (50).

### Example 2

Compound 2: 8-Hydroxy-3,3-dimethyl-1,2-dihydro-3H-naphtho[2,1-b]pyran-7,10-dione

A mixture of 8-hydroxy-3,3-dimethyl-3H-naphtho[2,1-b]pyran-7,10-dione (132 mg, 0.52 mmol) and platinum (IV) oxide (15 mg) in ethyl acetnte (15 mL) was stirred under an atmosphere of hydrogen for 7 h. The resulting mixture was stirred in air for 1 h then was filtered through a pad of diatomaecous earth. The pad was washed with ethyl acetate then the filtrate and washings were combined and cocentrated in vacuo to give a green solid (128 mg, 96%). Recrystallisation from ethyl acetate/hexanes using activated charcoal gave 8-hydroxy-3,3dimethyl-1,2-dihydro-3H-naphtho[2,1-b]pyran-7,10-dione m.p. 183.5-187°. δ (¹H) (300 MHz, CDCl₃) 1.37, s, 2×CH₃; 1.85, t, J 6.8 Hz, 2×H2; 3.30, t, J 6.8 Hz, 2×H1; 6.20, s, H9; 7.03, d, J 8.6 Hz, H5; 7.98, d, J 8.6 Hz, H6. m/z (ES⁺, 30 V) 259 (M+H, 77%), 174 (88), 159 (100).

### Example 3

Compound3: 8-Acetoxy-3,3-dimethyl-3H-naphtho[2,1-b]pyran-7,10-dione

Concentrated sulphuric acid (1 drop) was added to a stirred orange suspension of 8-hydroxy-3,3-dimethyl-3H-naphtho[2,1-b]pyran-7,10-dione (8.55 mg, 3.34 mmol) in acetic anhydride (10 mL) and the mixture was placed in an oil bath (oil bath temperature 100° C.). The mixture immediately became homogeneous red-black. After 10 min the mixture was cooled(ice/water bath) and water (50 mL) added. Products were extracted with ethyl acetate (150 mL), the organic phase separated, dried (Na₂SO₄) and filtered through a silica plug, washing the plug with ethyl acetate until no further colour eluted. The filtrate was concentrated in vacuo and the residue was dried overnight under vacuum to afford the title compound as a red solid (965 mg, 97%). ¹H NMR (300 MHz, CDCl₃) δ 1.48 (6H, s, 2×CH₃), 2.37 (3H, s, COCH₃), 5.94 (1H, d, J 10.5 Hz, H2), 6.34 (1 H, s, H9), 7.07 (1H, d, J 8,4 Hz, H5), 7.73 (1H, d, J 10.5 Hz, H1), 7.97, (1H, d, J 8.4 Hz, H6).

### Example 4

Compound 4: 7,8,10-triacetoxy-3,3-dimethyl-3H-naphtho[2,1-b]pyran

A stirred solution of 8-hydroxy-3,3-dimethyl-3H-naphtho[2,1-b]pyran-7,10-dione (110 mg, 0,93 mmol) in acetic anhydride (3 mL) and pyridine (4 mL) was heated in an oil bath at 60° C. for 15 min. Zinc powder (530 mg) was added in one portion and the mixture became pale yellow. After 15 min healing, the mixture was cooled to room temperature and filtered through a sinter (porosity 4), with ethyl acetate washings. The filtrate was poured onto ice/water (20 mL) and acidified with aqueous hydrochloric acid solution (2.0 M). The organic phase was separated and the aqueous phase washed with ethyl acetate (3×50 mL). The combined organic phases were dried (Na₂SO₄, filtered and concentrated in vacuo. The resulting solid was reerystallised from ethanol to afford the title compound as a colourless solid (96 mg, 58%). 1H NMR (300 MHz, CDCl₃) δ 1.46 (6H, s, 2×CH₃), 2.31 (3H, s, COCH3), 2.37 (3H, s, COCH3), 2.43 (3H, s, COCH3), 5.64 (1H, d, J 10.1 Hz, H2). 7.11 (1H, s, H9), 7.12 (1H, d, J 9.0 Hz, H6), 7.23 (1H, d, J 10.2 Hz, H1), 7.67 (1H, d, J 9.0 Hz, H5).

### Example 5

Compound 5: 9-Bromo-8-hydroxy-3,3-dimethyl-3H-naphtho[2,1-b]pyran-7,10-dione

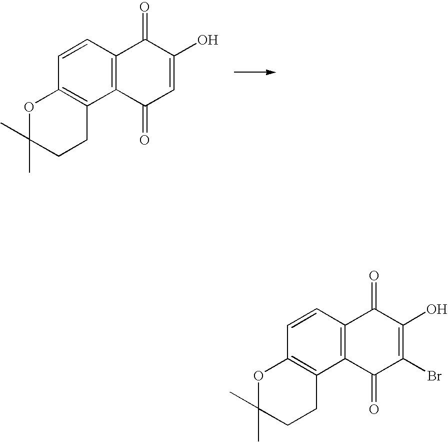

A solution of bromine (354 mg, 2.21 mmol) in dry dichloromethane (4 ml) was added dropwise to a cooled (0° C.) solution of 8-hydroxy-3,3-dimethyl-1,2-dihydro-3H-naphtho[2,1-b]pyran-7,10-dione (506 mg, 1.96 mmol) in dry dichloromethane (4 mL) containing 3 drops of glacial acetic acid. The cooling bath was removed and the mixture was stirred at room temperature for 20 min then concentrated in vacuo to afford 9-bromo-8-hydroxy-3,3-dimethyl-1,2-dihydro-3H-naphtho[2,1-b]pyran-7,10-dione as a bright orange powder (635 mg, 96%): m.p. 213-216° C. (Found; C, 53.5; H, 4.0. C₁₅H₁₃BrO₄ requires C, 53.4: H, 3.9 %). Vₘₐₓ 3316 m, 1660 s, 1642 s, 1364 s, 1286 s, 1262 s, 1174 m, 1114 s, 1048 s cm⁻¹. ¹H NMR (300 MHz, CDCl₃) δ 1.38 (6H, s, 2×CH₃), 1.87 (2H, t, J=6.8 Hz, H2), 3.32 (2H, t, J=6.8 Hz, H1), 7.06 (1H; d, J=8.6 Hz, H5), 8.00 (1H, d, J=8.6 Hz, H6). m/z (ESI⁺) 339 (M[⁸¹Br]+H), 337 (M[⁷⁹Br]+H).

### Example 6

Compound 6: 9-Bromo-8-hydroxy-3,3-dimethyl-3H-naphtho[2,1-b]pyran-7,10-dione
Compound 7: 2,9-dibromo-1,8-dihydroxy-3,3-dimethyl-1,2-dihydro-3H-naphtho[2,1-b]pyran-7,10-dione

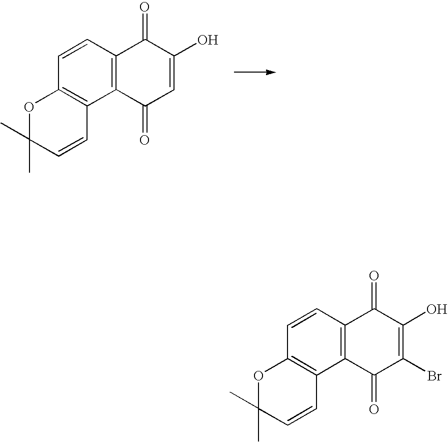

Sodium hydride (42 mg, 80% dispersion in oil, 1.40 mmol) was washed with dry hexane then the supernatant was removed. The residual solid was dried under a stream of nitrogen. A solution of 8-hydroxy-3,3-dimethyl-3H-naphtho[2,1-b]pyran-7,10-dione (327 mg, 1.23 mmol) in tetrahydrofuran (5 mL) was added and the resulting solution was stirred at room temperature for 10 min. This was cooled to 0° C. and a solution of bromine (265 mg, 1.66 mmol) in dry dichloromethane (3 mL) was added. Thie mixture was allowed to warm to room temperature and stirred for 20 min, after which the solvents were concentrated in vacuo to afford a brown residue. Flash chromatography (20-50% ethyl acetate/hexane with 1% glacial acetic acid) afforded 9-bromo-8-hydroxy-3,3dimethyl-3H-naphtho[2,1-b]pyran-7,10-dione as an orange-brown solid (43 mg, 10%). (Found: M+H, 334.9909, 336.9887. C₁₅H₁₂BrO₄⁺ requires 334.9919, 336.9900). ¹H NMR (300 MHz, CDCl₃,) δ 1.49 (6H, s, 2×CH₃), 5.99 (1H, d, J=10.2 Hz, H2), 7.05 (1H, d, J=8.4 Hz, H5), 7.75 (1H, bs, OH), 7.82 (1H, d, J=10.2 Hz, H1), 8.01 (1H, d, J=8.4 Hz, H6). m/z (ESI⁺ ) 337 (M[⁸¹Br]+H), 335 (M[⁷⁹Br]+H).

Also recovered from the flash chromatography column was crude compound 6 contaminated with a product of pyran ring bromination (compound 7). This was subjected to preparative HPLC (isocratic 60% A, 40% B) and gave a compound suspected of being 2,9-dibromo-1,8-dihydroxy-3,3-dimethyl-1,2-dilhydro-3H-naphtho[2,1-b]pyran-7,10-dione (3 mg, 0.5%), m.p. 202.5-205° (Found: M+H, 412.9011, 414.8981, 416.8961. [C₁₅H₁₃Br₂O₅-H₂O]⁺ requires 412.9024, 414.9005, 416.8987). Vₘₐₓ 3475 w, 1664 m, 1582 m, 1370 m, 1284 s, 1262 s, 1184 m, 1122 m, 1016 m cm⁻¹. δ (¹H) (300 MHz, CDCl₃) 1.59, s, CH₃; 1.66. s, CH₃; 4.42, d, J 3.8 Hz, H2; 4.73, bs, OH; 5.50, d, J 3.8 Hz, H1; 7.22, d, J 8.7 Hz, H5; 8.13, d, J 8.7 Hz, H6. m/z (ES⁺, 70 V) 457 (M[⁸¹Br][⁸¹Br]+Na, 13%), 455 (M[⁸¹Br][⁷⁹Br]+Na, 31), 453 (M[⁷⁹Br][⁷⁹Br]+Na, 22), 435 (M[⁸¹Br][⁸¹Br]+H, 7), 433 (M[⁸¹Br][⁷⁹Br]+H, 18), 431 (M[⁷⁹Br][⁷⁹Br]+H, 12), 417 (M[⁸¹Br][⁸¹Br]-H₂O+H, 16), 415 (M[⁸¹Br][⁷⁹Br]-H₂O+H, 36), 413 (M[⁷⁹Br][⁷⁹Br]-H₂O+H, 20), 336 (M-[⁷⁹Br+H₂O]+H, 100), 334 (M-[⁸¹Br+H₂O]H, 100).

### Example 7

Compound 8; 9-Bromo-3,3-dimethyl-8-(4-methylbenzenesulfonylnxy)-1,2-dihydro-3H-naphtho[2,1-b]pyran-7,10-dione
Compound 9: 9-Bromo-3,3-dimethyl-8-(4-methylbenzenesulfonyloxy)-3H-naphtho[2,1-b]pyran-7,10-dione

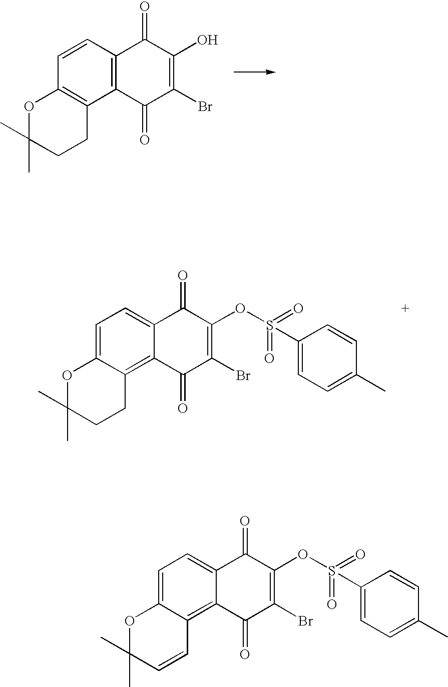

Pyridine (0.40 mL 4.95 mmol) was added to a cooled (0° C.), stirred solution of 9-bromo-8-hydroxy-3,3-dimethyl-1,2-dihydro-3H-naphtho[2,1-b]pyran-7,10-dione (550 mg, 1.63 mmol) in dry dichloromethane (10 mL) under nitrogen. A solution of 4-methylbenzenesulfonyl chloride (350 mg, 1.84 mmol) in dry dichloromethane (8 mL) was added dropwise, then stirring was continued for 1.5 h at 0° C. Diisopropylethylamine (2.5 mL, 14.4 mmol) was added and stirring was continued for a further 3 h when aqueous hydrochloric acid solution (2.0 M) was added. Products were extracted with dichloromethane and ethyl acetate, and the combined extracts dried, filtered and concentrated in vacuo to afford a brown solid (798 mg). Trituration of this with ethyl acetate/hexane gave a yellow solid (610 mg) which by ¹H NMR spectroscopy contained ˜10% of the unsaturated pyran (compound 9).

A sample of the above yellow solid (560 mg) was dissolved in ethyl acetate (30 mL) and platinum (IV) oxide (25 mg) was added. The resulting mixture was stirred under an atmosphere of hydrogen for 8.5 h after which it was filtered through a pad of Celite® and the filtrate was stirred in air at room temperature overnight. Concentration in vacuo gave a brown solid which contained ˜5% of the unsaturated pyran (compound 9) by ¹H NMR spectroscopy. The above hydrogenation was repeated on this solid using platinum (IV) oxide (69 mg) in ethyl acetate (35 mL) under hydrogen at room temperature overnight. The mixture was filtered through a pad of Celite® and the filtrate concentrated in vacuo to afford a brown residue (580 mg). This was dissolved in acetonitrile (45 mL) and a solution of ceric ammonium nitrate (617 mg) in water (20 mL) was added dropwise. The resulting mixture was stirred at room temperature for 2 h and water (35 mL) was added. The resulting precipitate was collected by filtration, washed with water, hexane and dried under vacuum for 2 h at 40° C. to give 9-bromo-3,3-dimethyl-8-(4-methylbenzenesulfonyloxy)-1,2-dihydro-3H-naphtho[2,1-b]pyran-7,10-dione as a brown solid (300 mg, 41%); m.p. 109-115° C. (Found: C, 53.6; H, 4.1. C₂₂H₁₉BrO₆S requires C, 53.8; H, 3.9 %). vₘₐₓ 1672 s, 1580 m, 1370 s, 1302 s, 1288 s, 1222 m, 1202 m, 1174 s, 994 s, 718 s cm⁻¹. ¹H NMR (300 MHz, CDCl₃) δ 1.37 (6H, s, 2×CH₃), 1.87 (2H, t, J=6.3 Hz, H2), 2.50 (3H, s, ArCH₃), 3.27 (2H, t, J=6.3 Hz, H1), 7.11 (1H, d, J=8.4 Hz, H5), 7.41 (2H, d, J=7.8 Hz, H3), 7.98 (3H, app d, J=8.1 Hz, 2×H2′ and H6). m/z (ESI⁺) 493 (M[⁸¹Br]+H), 491 (M[⁷⁹Br]+H).

### Example 8

Compound 9: 9-Bromo-3,3-dimethyl-8-(4-methylbenzenesulfonyloxy)-3H-naphtho[2,1-b]pyran-7,10-dione

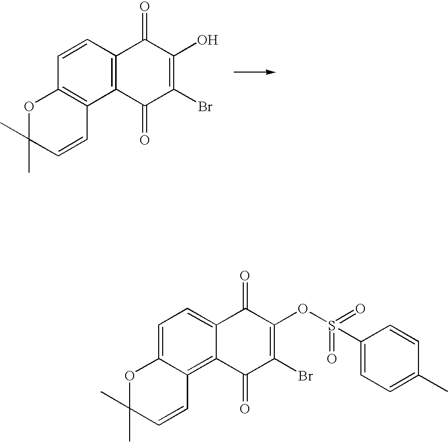

Pyridine (0.15 ml 1.85 mmol) was added to a cooled (0° C.), stirred solution of 9-bromo-8-hydroxy-3,3-dimethyl-3H-naphtho[2,1-b]pyran-7,10-dione (235 mg, 0.70 mmol) in dry dichloromethane (5 mL) under nitrogen. A solution of 4-methylbenzenesulfonyl chloride (0.148 mg, 0.78 mmol) in dry dichloromethane (4 mL) was added dropwise, and stirring was continued for 1 h at 0° C. Diisopropylethylamine (1.0 mL, 5.74 mmol) was added and stirring was continued for a further 3 h when aqueous hydrochloric acid solution (1.0 M) was added. Products were extracted with dichloromethane and ethyl acetate, and the combined extracts dried, filtered and concentrated in vacuo to afford a brown solid. Flash chromatography (ethyl acctate/hexane 3:7) afforded 9-bromo-3,3-dimethyl-8-(4-methylbenzenesulfonyloxy)-3H-naphtho[2,1-b]pyran-7,10-dione as a brown solid (97 mg, 28%): m.p. 167-168° C. (Found: M+H, 490.998, 488.999. C₂₂H₁₈BrO₆S⁺ requires 490.999, 489.001). vₘₐₓ 1672 s, 1388 m, 1288 s, 1218 m, 1172 s, 1112 m, 1018 m, 1004 m, 732 s, 706 m, 688 cm⁻. ¹H NMR (300 MHz, CDCl₃) δ 1.49 (6H, s, 2×CH₃), 2.50 (3H, s, ArCH₃), 6.00 (1H, d, J=10.5 Hz, H2), 7.10 (1H, d, J=8.4 Hz, H5), 7.41 (2H, d, J=8.1 Hz, H3′), 7.69 (1H, d, J=10.5 Hz, H1), 7.98 (2H, d, J=8.1 Hz, H2′), 8.00 (1H, d, J=8.4 Hz, H6). m/z (ESI⁺) 491 (M[⁸¹Br]+H), 490 (M[⁷⁹Br]+H+1)

### Example 9

Compound 10: 8-Bromo-3,3-dimethyl-9-(4-methylbenzenesulfonyloxy)-3H-naphtho[2,1-b]pyran-7,10-dione

### Step 1

3,3-Dimethyl-3H-naphtho[2,1-b]pyran-9-ol

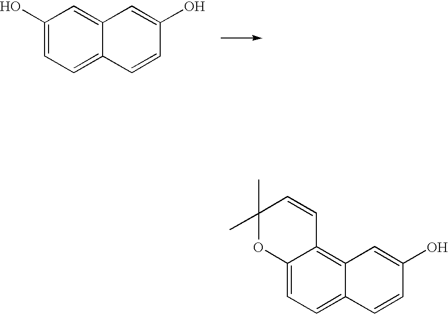

A mixture of 2,7-dihydroxynaphthalene (33.2 g, 207 mmol), 3-methyl-2-butenal (20.0 mL, 207 mmol) and pyridine (17.0 mL) was heated at 110° C. for 20 h under nitrogen. The mixture was cooled to room temperature and diluted with diethyl ether (150 mL). The organic phase was separated and washed successively with aqueous sulphuric acid solution (5%, 150 ml,), water (150 mL), aqueous sodium hydrogen carbonate solution (5%, 150 mL) and water (150 mL). The organic phase was dried, filtered and concentrated in vacuo to afford 3,3-dimethyl-3H-naphtho[2,1-b]pyran-9-ol as a buff coloured solid (43.1 g, 92%): ¹H NMR (300 MHz, CDCl₃) δ 1.48 (6H, s, 2×CH₃), 4.99 (1H, br s, OH), 5.67 (1H, d, J=10 Hz, H2), 6.84-6.93 (3H, m, H1, H5, H8), 7.23 (1H, d, J 2.3=Hz, H10), 7.55 (1H, d, J=8.8 Hz, H6), 7.63 (1H, d, J=8.8 Hz, H7). m/z (FAB, 3NBA/MeOH) 227 (M+H, 68%).

### Step 2

3,3-Dimethyl-3H-naphtho[2,1-b]pyran-9,10-dione

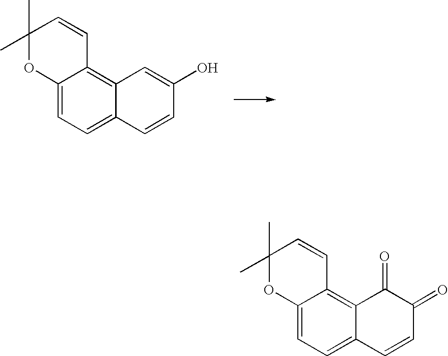

N,N′-Bis(salicylidene)ethylenediaminocobalt(II) hydrate (4.5 g, 14 mmol) was added to a stirred solution of 3,3-dimethyl-3H-naphtho[2,1-b]pyran-9-ol (43.2 g, 190 mmol) in acetonitrile (1.0 L) and oxygen bubbled through the mixture with reaction progress monitored by HPLC. Further portions of the catalyst (4.1 g, 3.4 g and 2.7 g) were added after 18.5 h, 24.5 h and 44.5 h respectively. After 112 h in total, HPLC showed no starting naphthol and the mixture was filtered though a silica pad (5×12 cm), washing the pad with ethyl acetate until no further red colour eluted. The filtrate was concentrated in vacuo and the resulting residue recrystallised from ethyl acetate/hexane to afford 3,3-dimethyl-3H-naphtho[2,1-b]pyran-9,10-dione as maroon needles (14.5 g, 32%): m.p. 109-110° C. ¹H NMR (300 MHz, CDCl₃) δ 1.46 (6H, s, 2×CH₃), 6.00 (1H, d, J=10.3 Hz, H2), 6.27 (1H, d, J=10 Hz, H8), 6.98 (1H, d, J=8.2 Hz, H5), 7.09 (1H, d, J=8.2 Hz, H6), 7.32 (1H, d, J=10.3 Hz, H1), m/z (FAB, 3NBA/MeOH) 242 (M+H+1, 52%).

### Step 3

9-Hydroxy-3,3-dimethyl-3H-naphtho[2,1-b]pyran-7,10-dione

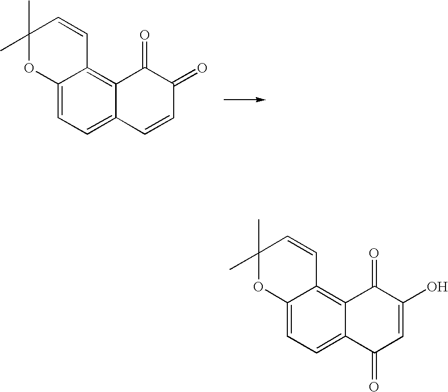

A solution of 3,3-dimethyl-3H-naphtho[2,1-b]pyran-9,10-dione (14.5 g, 60.3 mmol) in toluene (850 mL) was washed twice with a solution of sodium dithionlte (84 g) in water (850 mL). The resulting pale yellow solution was then added to a solution of potassium tert-butoxide (37.0 g, 330 mmol) in tert-butanol (370 mL) saturated with oxygen. The resulting mixture was stirred at ambient temperature for 30 min. after which aqueous hydrochloric acid solution (1.0 M, 250 mL) was added. The organic phase was separated and extracted with saturated aqueous sodium hydrogen carbonate (3×250 mL). The combined aqueous extracts were cooled (ice/water bath) and acidified with concentrated hydrochloric acid. The resulting precipitate was collected by filtration and washed with water (1.0 L) and dried under vacuum for 2 h at 40° C. to afford 9-hydroxy-3,3-dimethyl-3H-naphtho[2,1-b]pyran-7,10-dione as an orange solid (7.10 g, 46%): ¹H NMR (300 MHz, CDCl₃) δ 1.47 (6H, s, 2×CH₃), 5.99 (1H, d, J=10.4 Hz, H2), 6.25 (1H, s, H8), 7.11 (1H, dd, J=8.4, 0.6 Hz, H5), 7.47 (1H, s, OH), 7.76 (dd, J=10.4, 0.6 Hz, H1), 7.97 (1H, d, J=8.5 Hz, H6. m/z (FAB, 3NBA/MeOH) 258 (M+H+1, 50%), 257 (M+H, 100).

### Step 4

8-Bromo-3,3-dimethyl-9-(4-methylbenzenesulfonyloxy)-3H-naphtho[2,1-b]pyran-7,10-dione

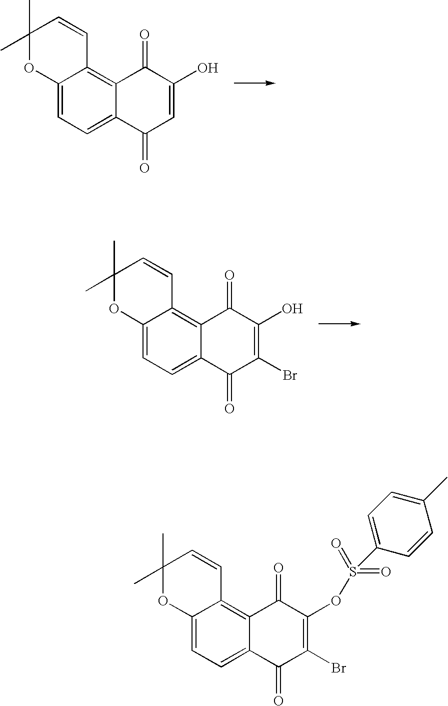

Sodium hydride (12 mg, 80% dispersion in oil, 0.40 mmol) was added to a solution of 9-hydroxy-3,3-dimethyl-3H-naphtho[2,1-b]pyran-7,10-dione (100 mg, 0.39 mmol) in tetrahydrofuran (2 mL) and the mixture was stirred at room temperature for 10 min. The suspension was cooled to 0° C., and a solution of bromine (70 mg, 0,44 mmol) in dichloromethane (1 mL) was added. The orange solution was allowed to warm to room temperature then stirred for 20 min. Aqueous hydrochloric acid solution (1.0 M) was added and the product was extracted with dichloromethane and ethyl acetate. The combined organic phases were dried, filtered and concentrated in vacuo to give a brown residue (129 mg, 99%).

Pyridine (25 μL, 0.31 mmol) was added to a cooled (0° C.). stirred solution of the above 8-bromo-9-hydroxy-3,3-dimethyl-3H-napho[2,1-b]pyran-7,10-dione (25 mg, 0.07 mmol) in dry dichloromethane (1 mL). A solution of 4-methylbenzenesulfonyl chloride (17 mg, 0.09 mmol) in dry dichloromethane (1 mL) was added dropwise, then stirring was continued for 1 h at 0′ C. Diisopropylethylamine (115 μL, 0.66 mmol) was added and stirring was continued for a further 3 h when aqueous hydrochloric acid solution (1.0 M) was added and the mixture extracted with dichloromethane and ethyl acetate. The combined organic phases were dried, filtered and concentrated in vacuo to afford 8-bromo-3,3-dimethyl-9-(4-methylbenzenesulfonyloxy)-3H-naphtho[2,1-b]pyran-7,10-dione as a brown solid (35 mg, 95%): m.p. 181.5-184° C. (Found: C, 54.0; H, 3.5. C₂₂H₁₇BrO₆S requires C, 54.0; H, 3.5 %). vₘₐₓ 1678 m, 1382 s, 1294 s, 1285 s, 1183 m, 1128 m, 1062 m, 986 m, 806 s, 682 s, 564m cm⁻¹, ¹H NMR (300 MHz, CDCl₃) δ 1.49 (6H, s, 2×CH₃), 2.50 (3H, s, ArCH₃), 5.98 (1H, d, J=10.5 Hz, H2), 7.09 (1H, d, J=8.4 Hz, H5), 7.41 (2H, d, J=8.3 Hz, H3), 7.70 (1H, d, J=10.5 Hz, H1), 7.98 (2H, d, J=8.3 Hz, H2), 8.04 (1H, d, J 8.4 Hz, H6). m/z (ESI⁺) 491 (M[⁸¹Br]+H), (M[⁷⁹Br]+H)

### Example 10

Compound 11: 8-Bromo-3,3-dimethyl-9-(4-methylbenzenesulfonyloxy)-1,2-dihydro-3H-naphtho[2,1-b]pyran-7,10-dione

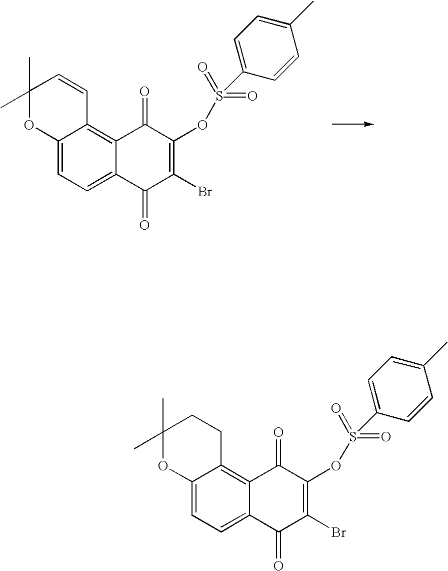

Platinum (IV) oxide (25 mg) was added to a solution of 8-bromo-3,3-dimethyl-9-(4-methylbenzenesulfonyloxy)-3H-naphtho[2,1-b]pyran-7,10-dionc (199 mg, 0.41 mmol) in ethyl acetate (8 mL) and the resulting suspension was stirred under an atmosphere of hydrogen for 28 h. The reaction was filtered through a pad of Celite® and the filter cake was washed with ethyl acetate and dichloromethane. The filtrate and washings were combined, dried, filtered and concentrated in vacuo to afford a brown residue which was dissolved in acetonitrile (20 mL) and cooled to 0° C. A solution of ceric ammonium nitrate (200 mg, 0.36 mmol) in water (6 mL) was then added and the mixture was allowed to warm to room temperature and stirred for 2 h, after which it was diluted with water. Products were extracted with dichloromethane, and the extract dried, filtered and concentrated in vacuo to afford 8-bromo-3,3-dimethyl-9-(4-methylbenzenesulfonyloxy)-1,2-dihydro-3H-naphtho[2,1-b]pyran-7,10-dione as an orange-brown solid (193 mg, 97%): m.p. 168-169.5° C. (Found: C, 53.6; H, 3.8. C₂₂H₁₉BrO₆S requires C, 53.8; H, 3.9%). vₘₐₓ 1630 s, 1668 m, 1620 m, 1578 w, 1566 w, 1382 s, 1290s cm⁻¹. ¹H NMR (300 MHz, CDCl₃) δ 1.37 (6H, s, 2×CH₃), 1.87 (2H, t, J=6.2 Hz, H2), 2.50 (3H, s, ArCH₃), 3.27 (2H, t, J=6.2 Hz, H1), 7.10 (1H, d, J 8.6=Hz, H5), 7.41 (2H, d, J=7.7 Hz, H3′), 7.97 (2H, d, J=7.7 Hz, H2′), 8.03 (1H, d, J=8.6 Hz, H6). m/z(ESI⁺)(M[⁸¹Br]+H), 491 (M[⁷⁹Br]+H).

### Example 11

Compound 12: 8,9-Dichloro-3,3-dimethyl-1,2-dihydro-3H-naphtho[2,1-b]pyran-7,10-dione

### Step 1

9-Hydroxy-3,3-dimethyl-1,2-dihydro-3H-naphtho[2,1-b]pyran-7,10-dione

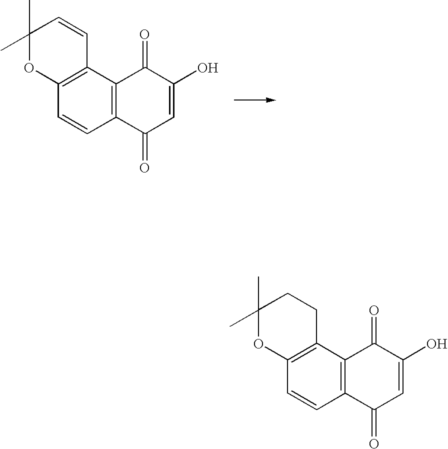

A mixture or 9-hydroxy-3,3-dimethyl-3H-naphtho[2,1-b]pyran-7,10-dione (1.20 g, 4.6 mmol) and platinum(IV) oxide (125 mg) in ethyl acetate (30 mL) was stirred under hydrogen for 3.5 h. The dark mixture was allowed to stir exposed to air for 30 min before filtration through a plug of Celite®. Concentration of the filtrate in vacuo gave a yellow residue which was subjected to flash chromatography (ethyl acctate/hexane 7:3 with 1% glacial acetic acid) followed by recrystallisation from ethyl acetatc/hexane to give 9-hydroxy-3,3-dimethyl-1,2-dihydro-3H-naphtho[2,1-b]pyran-7,10-dione as yellow needles (948 mg, 79%): m.p. 155° C. (subl.), >177° C. (dec). vₘₐₓ 3328, 3148, 3103, 1656, 1628, 1566 cm⁻¹. ¹H NMR (300 MHz, CDCl₃) δ 1.38 (6H, s, 2×CH₃), 1.89 (2H, t, J=6.5 Hz, H2), 3.29 (2H, t, J=6.5 Hz, H3), 6.25 (1H, s, H8), 7.13 (1H, d, J=8.5 Hz, H5), 7.47 (1H, s, OH), 7.97 (1H, d, J=8.5 Hz, H6). m/z (FAB, 3NBA/MeOH) 261 (M+H+2, 11%), 260 (M+H+1, 27%), 259 (M+H, 51).

### Step 2

9-Chloro-3,3-dimethyl-1,2-dihydro-3H-naphtho[2,1-b]pyran-7,10-dione

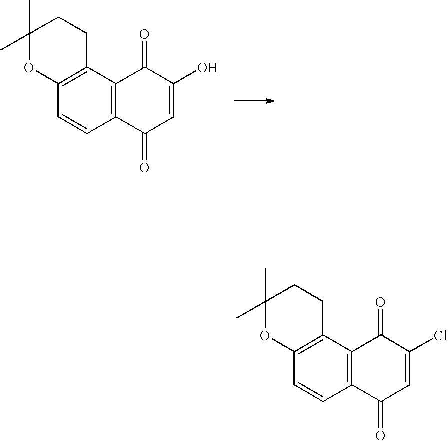

9-Hydroxy-3,3-dimethyl-1,2-dihydro-3H-naphtho[2,1-b]pyran-7,10-dione (1.1 g, 4.3 mmol) was dissolved in dichloromethane (20 mL) and thionyl chloride (15 mL). The reaction was stirred at room temperature for 24 h and the volatiles were removed in vacuo.

The residue was dissolved in ethyl acetate (30 mL), washed with water (30 mL), dried, filtered and concentrated in vacuo. The residue was purified by flash chromatography (ethyl acetate/hexane 5:95) followed by recrystallisation from ethanol to give 9-chloro-3,3-dimethyl-1,2-dihydro-3H-naphtho[2,1-b]pyran-7,10-dione as orange needles (633 mg, 53%): m.p. 140-2° C. (Found: C, 64.9; H, 4.8. C₁₅H₁₃ClO₃ requires C, 65.1; H, 4.7%). λₘₐₓ (log ε) 216, 268, 350 sh, 412 nm (4.35, 4.25, 3.23, 3.44). vₘₐₓ 3700-3300 s br, 3070 w, 3000 w, 1680 s, 1660 s, 1620 m, 1590 m, 1580 s cm⁻¹. ¹H NMR (300 MHz, CDCl₃) δ 1.38 (6H, s, 2×CH₃), 1.88 (2H, t, J=7.0 Hz, H2), 3.29 (2H, t, J=7.0 Hz, H1), 7.12 (1H, d, J=8.5 Hz, H5), 7.12 (1H, s, H8), 7.95 (1H, d, J=8.5 Hz, H6). m/z (FAB, 3NBA) 280 (M[³⁷Cl]+H+1, 14%), 279 (M[³⁷Cl]+H, 40), 278 (M[³⁵Cl]+H+1, 49), 277 (M[³⁵Cl]+H, 100), 276 (44), 233 (17).

### Step 3

8,9-Dichloro-3,3-dimethyl -1,2-dihydro-3H-naphtho[2,1-b]pyran-7,10-dione

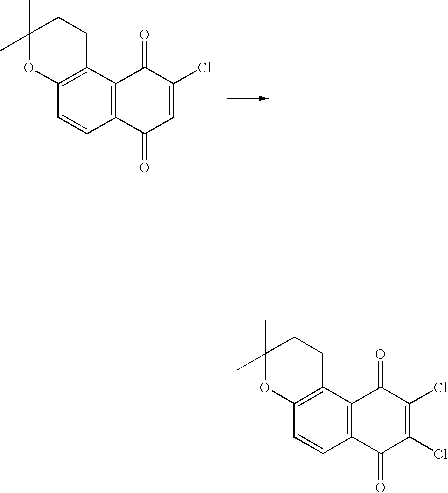

Chlorine gas was bubbled through a solution of 9-chloro-3,3-dimethyl-1,2-dihydro-3H-naphtho[2,1-b]pyran-7,10-dione (633 mg, 2.3 mmol) in glacial acetic acid (50 mL) containing concentrated hydrochloric acid (5 drops) at 70° C. for 5 min. The reaction was stirred for 55 min at 70° C., cooled to room temperature and concentrated in vacuo. The residue was purified by flash chromatography (toluene/hexane 7:3) to give 8,9-dichloro-3,3-dimethyl -1,2-dihydro-3H-naphtho[2,1-b]pyran-7,10-dione as an orange solid (508 mg, 71%). A sample of this material was recrystallised from ethanol to give orange micro-crystals: m.p. 158-60° C. (Found: C, 57.9; H, 3.7. C₁₅H₁₂Cl₂O₃ requires C, 57.9; H, 3.9%). λₘₐₓ (log ε) 220, 275, 290 sh, 350 sh, 412 nm (4.32, 4.19, 4.01, 3.29, 3.38). vₘₐₓ 3700-3330 m br, 3050 m, 2950 m, 1700 s, 1680 m, 1600 m, 1580 s cm⁻¹. ¹H NMR (300 MHz, CDCl₃) δ 1.38 (6H, s, 2×CH₃), 1.88 (2H, t, J=7.0 Hz, H2), 3.27 (2H, t, J=7.0 Hz, H1), 7.12 (1H, d, J=8.5 Hz, H5), 8.04 (1H, d, J=8.5 Hz, H6). m/z (FAB, 3NBA) 315 (M[³⁷Cl₂]+H, 16%), 314 (M[³⁷Cl³⁵Cl]+H+1, 32), 313 (M[³⁷Cl³⁵Cl]+H, 74), 312 (M[³⁵Cl₂]+H+1, 64), 311 (M[³⁵Cl₂]+H, 100), 310 (42), 309 (16).

### Example 12

Compound 13: Sodium 3,3-dimethyl-7,10-dioxo-7,10-dihydro-3H-benzo[f]chromen-8-olate or Sodium 3,3-dimethyl-7,8-dioxo-7,8-dihydro-3H-benzo[f]chromen-10-olate

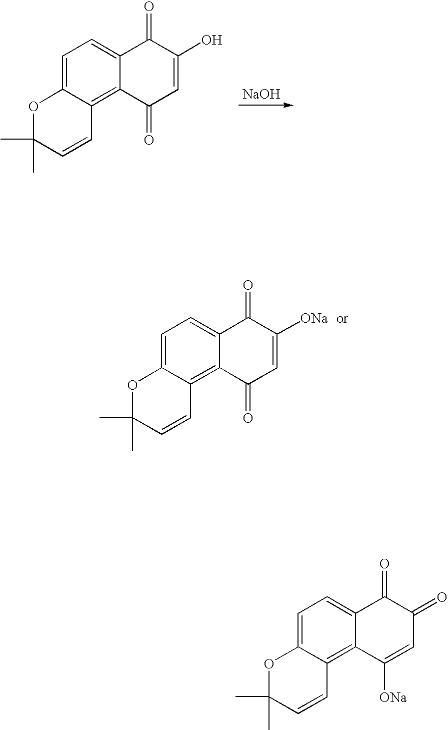

Aqueous sodium hydroxide (2.0 M, 3.38 mL, 6.75 mmol) was added dropwise to a stirred orange suspension of compound 1 (1.73 g, 6.75 mmol) in methanol (10 ml). The mixture became homogeneous red. After 30 min volatiles were removed in vacuo and the resulting red residue dissolved in water (150 mL), filtered (porosity 4 sinter) and freeze dried for 48 h. Compound 13 was obtained as a red solid (1.80 g, 96%): ¹H NMR (300 MHz, D⁶DMSO) δ 1.38 (6H, s, 2×CH₃), 5.26 (1H, s, H9), 5.82 (1H, d, J=10.2 Hz, H2), 6.83 (1H, d, J=8.4 Hz, H6), 7.64 (1H, d, J=8.4 Hz, H5), 8.14 (1H, d, J=10.2 Hz, H1), m/z (ES⁺, 30 V) 257 (M−Na+H, 100%); HPLC 100%/4.74 min.

The ¹H NMR can also be run in D₂O, compound 13 is fully soluble in water at 10 mg/mL

### HPLC Conditions

Performed on Water 2690 Alliance System, using a Waters C18 5 μm Symmetry Column (Part # WAT046980) and a flow rate of 0.7 mL/min. Column temperature of 30° C. and measured at λ=254nM.

### Buffers

Buffer A: 100% water
Buffer B: 100%
Buffer C: 2% aqueous formic acid
   Gradient (linear gradient curve “6”)

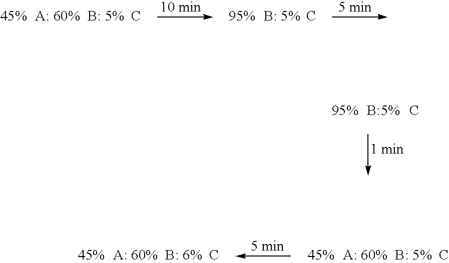

### Example 13

Compound 14: 8-Hydroxy-3-methyl-3-phenyl-3H-benzo[f]chromene-7,10-dione

### Step 1

3-Methyl-3-phenyl-3H-benzo[f]chromen-8-ol

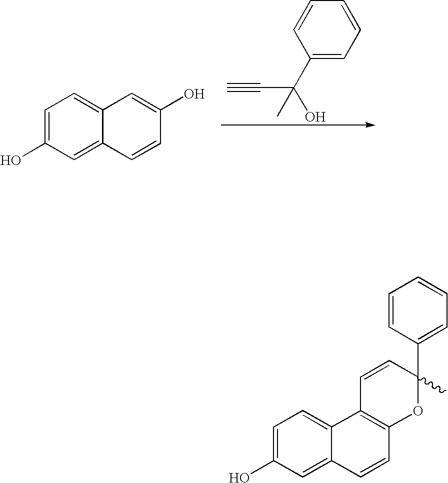

A stirred suspension or 2,6-dihydroxy naphthalene (716 mg, 4.47 mmol) in toluene (200 mL) was heated to reflux (oil bath temperature 160° C.). After 1 h, the mixture was homogeneous and p-toluenc sulphonic acid hydrate (54 mg, 0.40 mmol) was added followed by a solution of 2-phenyl-but-3-yn-2-ol (588 mg, 4.02 mmol) in toluene (50 mL) over 20 min while maintaining reflux. TLC (ethyl acetate/hexane 1:4) after 4 h showed very faint 2,6-dihydrxy naphthalene. After a further 2 h, the mixture was cooled to room temperature and washed with aqueous sodium hydroxide solution (10%, 400 mL). The organic phase was diluted with ethyl acetate (100 mL), dried (Na₂SO₄), filtered and concentrated in vacuo to afford a black semi-solid (804 mg). This was dissolved in ethyl acetale/hexane/dichloromethane/methanol (1 mL:4 mL:1 mL:1 mL) and subjected to flask chromatography, eluting with ethyl acetate/hexane 1:4. 3-Methyl-3-phenyl-3H-benzo[f]chromen-8-ol was obtained as a brown solid (230 mg, 18): MS (ESI⁻) m/z 287 (M−1). HPLC99.3 %/7.58 min

### Step 2

3-Methyl-3-phenyl-3H-benzo[f]chromene-7,8-dione

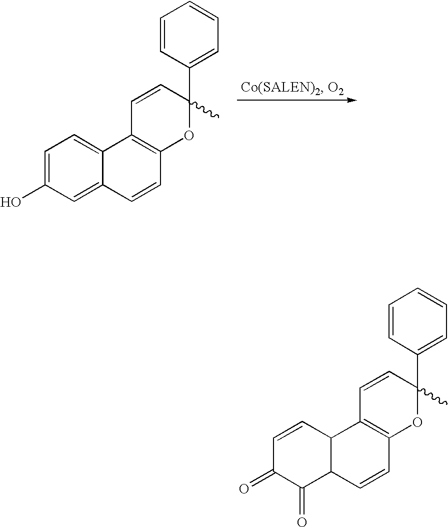

Co(SALEN)₂ (23 mg) was added in one portion to a stirred homogenous yellow solution of 3-methyl-3-phenyl-3H-benzo[f]chromen-8-ol (217 mg, 0.75 mmol) in acetonitrile (3 mL). Oxygen was bubbled through the mixture and after 90 min the mixture was filtered through a silica plug, washing the plug with ethyl acetate until no further colour eluted. Volatiles were removed in vacuo to afford 3-methyl-3-phenyl-3H-benzo[f]chromene-7,8-dione as an orange solid (228 mg, 100%): ¹H NMR (300 MHz, CDCl₃) δ 1.86 (3H, s, CH₃), 6.25 (1H, d, J=10.2 Hz, H2), 6.43 (1H, d, J=10.5 Hz, H9), 6.85 (1H, d, J=10.2 Hz, H1), 6.97 (1H, d, J=8.4 Hz, H6), 7.35 (5, m, ArH), 7.72 (1H, d, J=10.5 Hz, H9), 7.99 (1H, d, J=8.4Hz, H5) MS (ESI⁺) m/z 303 (M+1).

### Step 3

8-Hydroxy-3-methyl-3-phenyl-3H-benzo[f]chromene-7,10-dione

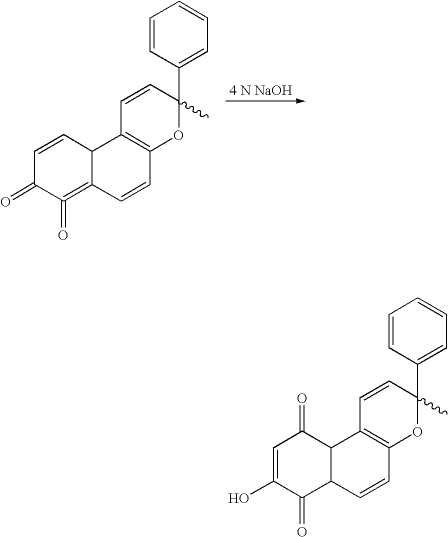

Aqueous sodium hydroxide solution (4 M, 5 mL) was added to stirred orange suspension of 3-methyl-3-phenyl-3H-benzo[f]chromene-7,8-dione (32 mg, 0.11 mmol) in ethanol (5 mL) and the mixture became homogeneous brown. After 1 h, the mixture was cooled (ice/water bath) and acidified to pH˜2.0 (5.0 M aqueous hydrochloric acid solution). The resulting orange suspension was stirred for 20 min in the cooling bath then at 10 min at room temperature. The precipitate was collected by filtration and washed with water (30 mL) then dried overnight under vacuum to afford 8-hydroxy-3-methyl-3-phenyl-3H-benzo[f]chromene-7,10-dione as an orange solid (27 mg, 82%): ¹H NMR (300 MHz, CDCl₃) δ 1.84 (3H, s, CH₃), 6.22 (1H, s, H9), 6.26 (1H, d, J=10.5 Hz, H2), 7.14 (1H, d, J=8.7 Hz, H6), 7.20-7.45 (5H, m, ArH), 8.05 (2H, m, H5 and H1). MS (ESI⁻) m/z 317 (M−1). HPLC 100 %/7.12 min.

### Example 14

Compound 15: 8-Hydroxy-3,3-diphenyl-3H-benzo[f]chromene-7,10-dione

### Step 1

3,3-Diphenyl-3H-benzo[f]chromen-8-ol

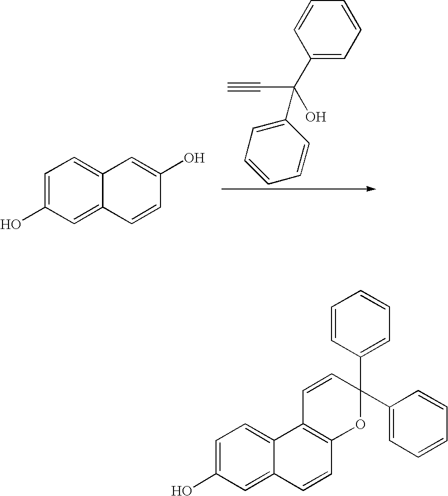

A stirred suspension of 2,6-dihydroxy naphthalene (502 mg, 3.13 mmol) in toluene (200 mL) was heated to reflux (oil bath temperature 130° C.). After 1 h, the mixture was homogeneous end p-toluene sulphonic acid hydrate (54 mg, 0.28 mmol) was added followed by a solution of 1,1-diphenyl-prop-2-yn-1-ol (588 mg, 2.82 mmol) in toluene (40 mL) over 30 min while maintaining reflux. After 2 d, the mixture was cooled to room temperature and washed with aqueous sodium hydroxide solution (10%, 400 mL). The organic phase was diluted with ethyl acetate (200 mL), dried (Na₂SO₄, filtered and concentrated in vacuo to afford a black solid. This was dissolved in chloroform/hexane (5 mL:5 ml) and subjected to flash chromatography, eluting with chloroform/hexane 1:1 then neat chloroform. 3,3-Diphenyl-3H-benzo[f]chromen-8-ol was obtained as a brown solid (213 mg, 19%): MS (ESI⁻) m/z 287 (M−1). HPLC 80.3%/8.89 min

### Step 2

3,3-Diphenyl-3H-benzo[f]chromene-7,8-dione

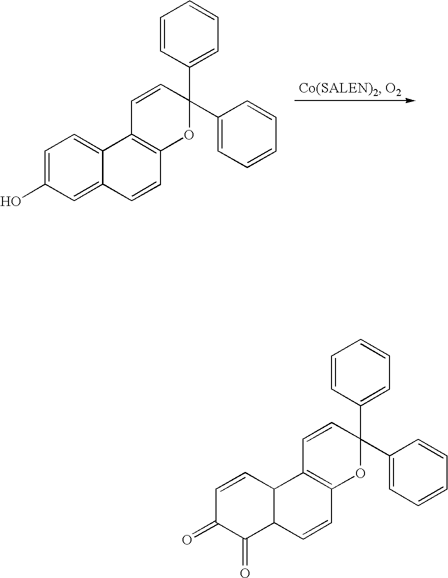

Co(SALEN)₂ (22 mg) was added in one portion to a stirred homogenous yellow solution of 3,3-diphenyl-3H-benzo[f]chromen-8-ol (213 mg, 0.61 mmol) in acetonitrile (3 mL). Oxygen was bubbled through the mixture and after 3 h the mixture was fillered through a silica plug, washing the plug with ethyl acetate until no further colour eluted. Volatiles were removed in vacuo to afford brown solid which was dissolved in dichloromethane and the solution subjected to flash chromatography, eluting with neat dichloromethane. 3,3-Diphenyl-3H-benzo[f]chromene-7,8-dione was obtained as an orange solid (32 mg, 14%). MS (ESI⁺) m/z 365 (M+1).

### Step 3

8-Hydroxy-3,3-diphenyl-3H-benzo[f]chromene-7,10-dione

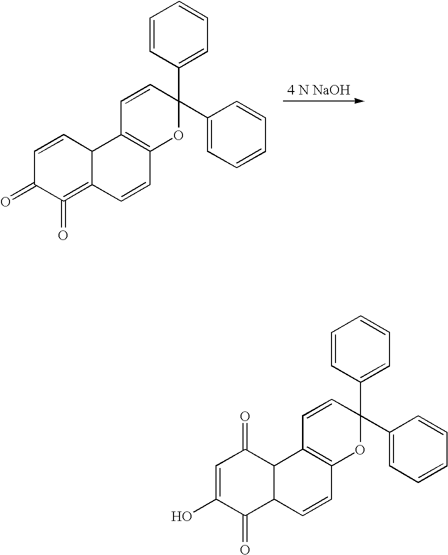

Aqueous sodium hydroxide solution (4 M, 3 mL) was added to stirred orange suspension of 3,3-diphenyl-3H-benzo[f]chromene-7,8-dione (22 mg, 0.06 mmol) in ethanol (3 mL) and the mixture became homogeneous brown. After 30 min, the mixture was cooled (ice/water bath) and acidified to pH˜2.0 (5.0 M aqueous hydrochloric acid solution). The resulting orange suspension was stirred for 20 min in the cooling bath then at 10 min at room temperature. The precipitate was collected by filtration and washed with water (15 mL). This residue was dissolved in ethyl acetate (5 mL) and filtered though a silica plug, eluting the plug with ethyl acetate (150 mL) then ethyl acetate/acetic acid (30:1), collecting 3×15 mL fractions. Fractions 1 and 2 were combined and concentrated in vacuo to afford 8-hydroxy-3,3-diphenyl-3H-benzo[f]chromene-7,10-dione as an orange solid (6 mg, 24%). ¹H NMR (300 MHz, CDCl₃) δ 6.23 (1H, s, H9), 6.45 (1H, d, J=10.4 Hz, H2), 7.20 (1H, d, J=8.4 Hz, H6), 7.40 (10H, m, ArH), 8.00 (1H, d, J=8.4 Hz, H5), 8.16 (1H, d, J=10.4 HZ, H1). MS (ESI⁺) m/z 381 (M+1). HPLC 86.5%/8.81 min.

### Antiviral Activity

Tests of antiviral activity were performed in 2.2.15 human hepatoma cells infected with hepatitis B according to the method of Korba and Gerin, Antiviral Research, 19, 55-70 (1992). Briefly, cells were seeded into 96 well plates and cell media containing various concentrations of the compounds was added. Media was changed daily for 9 days and fresh media containing compound was added each day. On the 10^{th} day, viral DNA in the supernatant was measured and the reduction in the amount of virus in the supernatant was calculated compared to cells incubated without drug. Six separate replicates were performed for each drug concentration. The effective concentration for 50% and 90% inhibition of the replication of the virus was determined from dose response curves. Results for some compounds of the invention are shown in Table 1.

TABLE 1

| Test Compound | EC50 μM | EC90 μM |
|---|---|---|
| Compound 1 | 0.6 | 3.4 |
| Compound 2 | 1.1 | 1.3 |

Antiviral activity was also examined in HepG2 hepatoma cells infected with HBV containing mutations associated with resistance to lamivudine (3TC). Two cell lines containing an L180M mutation in the HBV DNA polymerase, and a double L180M/M204V mutation were used. Cells were plated out in six well plates and allowed to attach overnight. Next day, the culture medium was replaced with either medium alone or medium containing the desired concentration of antiviral compound. Media was changed for fresh medium with or without antiviral compound on day 3. On day 5, supernatant and cell lysatcs were analysed for levels of HBV core protein by non-denaturing Western blot using an anti-HBV core antibody.

Results for some of the compounds are shown in Table 2 where a 50% reduction or more in measured level of the viral core protein compared to controls at a compound concentration of greater than 50 μmolar is designated +50% core reduction at less than 50 μmolar is designated ++ and 50% core reduction at less than 10 μmolar compound concentration is designated +++.

TABLE 2

| Test Compound | ≧50% core reduction |
|---|---|
| Compound 1 | +++ |
| Compound 5 | ++ |
| Compound 6 | + |
| Compound 13 | + |

1. A method of treatment or prophylaxis of hepatitis B virus in a subject comprising administering to said subject an effective amount of a compound of formula (1) or a pharmaceutically acceptable derivative, salt or prodrug thereof: 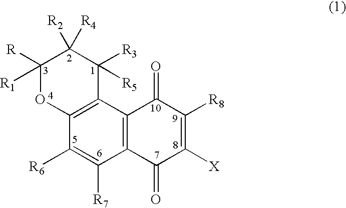
wherein X is OH, OR₉ or halo;
R and R₁ are independently selected from H, C₁₋₆ alkyl, C₂₋₆ alkenyl, C₂₋₆ alkynyl, C₃₋₆ cycloalkyl, aryl, or together with the carbon atom to which they are attached form a saturated or unsaturated C₃₋₆ carbocyclic ring;
R₂ and R₃ are independently selected from H, C₁₋₆ alkyl, C₂₋₆ alkenyl, C₂₋₆ alkynyl, C₃₋₆ cycloalkyl or together with the bond between the carbon atoms to which they are attached form a double bond;
R₄ and R₅ are independently selected from H, C₁₋₆ alkyl, C₂₋₆ alkenyl, C₂₋₆ alkynyl, C₃₋₆ cycloalkyl, OH, OR₉, halo or NR₁₀R₁₀ or together with the bond between the carbon atoms to which they are attached form a double bond;
R₆ and R₇ are independently selected from H, C₁₋₆ alkyl, C₂₋₆ alkenyl, C₂₋₆ alkynyl, C₃₋₆ cycloalkyl, OH or OR₉;
R₈ is independently selected from H, C₁₋₆ alkyl, C₂₋₆ alkenyl, C₂₋₆ alkynyl, C₃₋₆ cycloalkyl, OH, OR₉ or halo; R₉ is C₁₋₆ alkyl, C₂₋₆ alkenyl, C₂₋₆ alkynyl, C₃₋₆ cycloalkyl, aryl, C(═O)R₁₁ or S(O)₂R₁₂ or OR₉ is an amino acid residue;
each R₁₀ is independently selected from H and C₁₋₆ alkyl;
R₁₁ is C₁₋₂₁ alkyl, C₂₋₂₁ alkenyl, C₂₋₂₁ alkynyl, C₃₋₆ cycloalkyl, C₃₋₆ cycloalkyl C₁₋₈ alkyl, aryl or aryl C₁₋₆ alkyl; and
R₁₂is C₁₋₆ alkyl, C₂₋₆ alkenyl, C₂₋₆ alkynyl or aryl.

2. A method according to claim 1 wherein the compound of formula (1) is a compound of formula (2): 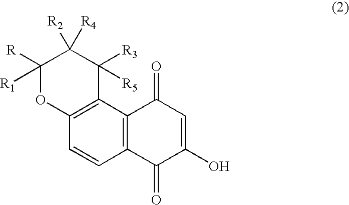
R and R₁ are independently selected from H, C₁₋₆ alkyl, C₂₋₆ alkenyl, C₂₋₆ alkynyl, C₃₋₆ cycloalkyl, aryl, or together with the carbon atom to which they are attached form a saturated or unsaturated C₃₋₆ carbocyclic ring;
R₂ and R₃ are independently selected from H, C₁₋₆ alkyl, C₂₋₆ alkenyl, C₂₋₆ alkynyl, C₃₋₆ cycloalkyl or together with the bond between the carbon atoms to which they are attached form a double bond;
R₄ and R₅ are independently selected from H, C₁₋₆ alkyl, C₂₋₆ alkenyl, C₂₋₆ alkynyl, C₃₋₆ cycloalkyl, OH, OR₉, halo or NR₁₀R₁₀ or together with the bond between the carbon atoms to which they are attached form a double bond;
R₉ is C₁₋₆ alkyl, C₂₋₆ alkenyl, C₂₋₆ alkynyl, C₃₋₆ cycloalkyl, aryl, C(═O)R₁₁ or S(O)₂R₁₂ or OR₉ is an amino acid residue;
each R₁₀ is independently selected from H and C₁₋₆ alkyl;
R₁₁ is C₁₋₂₁ alkyl, C₂₋₂₁ alkenyl, C₂₋₂₁ alkynyl, C₃₋₆ cycloalkyl, C₃₋₆ cycloalkyl C₁₋₆ alkyl, aryl or aryl C₁₋₆ alkyl; and
R₁₂ is C₁₋₆ alkyl, C₂₋₆ alkenyl, C₂₋₆ alkynyl or aryl.

3. The method of claim 1 wherein the compound of formula (1) is selected from the group consisting of:
8-hydroxy-3,3-dimethyl-3H-naphtho[2,1-b]pyran-7,10-dione,
8-hydroxy-3,3-dimethyl-1,2-dihydro-3H-naphtho[2,1-b]pyran-7,10-dione,
9-bromo-8-hydroxy-3,3-dimethyl-1,2-dihydro-3H-naphtho[2,1-b]pyran-7,10-dione,
9-bromo-8-hydroxy-3,3-dimethyl-3H-naphtho[2,1-b]pyran-7,10-dione,
9-bromo-3,3-dimethyl-8-(4-methylbenzenesulfonyloxy)-1,2-dihydro-3H-naphtho[2,1-b]pyran-7,10-dione,
9-bromo-3,3-dimethyl-8-(4-methylbenzenesulfonyloxy)-3H-naphtho[2,1-b]pyran-7,10-dione,
8-acetoxy-3,3-dimethyl-3H-naphtho[2,1-b]pyran-7,10-dione,
2,9-dibromo-1,8-dihydroxy-3,3-dimethyl-1,2-dihydro-3H-naphtho[2,1-b]pyran-7,10-dione,
8,9-dichloro-3,3-dimethyl-1,2-dihydro-3H-naphtho[2,1-b]pyran-7,10-dione,
7,8,10-triacetoxy-3,3-dimethyl-3H-naphtho[2,1-b]pyran,
9-Bromo-8-hydroxy-3,3-dimethyl-3H-naphtho[2,1-b]pyran-7,10-dione.
9-Bromo-8-hydroxy-3,3-dimethyl-3H-naphtho[2,1-b]pyran-7,10-dione.
9-Bromo-3,3-dimethyl-8-(4-methylbenzenesulfonyloxy)-1,2-dihydro-3H-naphtho[2,1-b]pyran-7,10-dione.
9-Bromo-3,3-dimethyl-8-(4-methylbenzenesulfonyloxy)-3H-naphtho[2,1-b]pyran-7,10-dione,
8-Bromo-3,3-dimethyl-9-(4-methylbenzenesulfonyloxy)-3H-naphtho[2,1-b]pyran-7,10-dione,
8-Bromo-3,3-dimethyl-9-(4-methylbenzenesulfonyloxy)-1,2-dihydro-3H-naphtho[2,1-b]pyran-7,10-dione,
8,9-Dichloro-3,3-dimethyl -1,2-dihydro-3H-naphtho[2,1-b]pyran-7,10-dione,
Sodium 3,3-dimethyl-7,10-dioxo-7,10-dihydro-3H-benzo[f]chromen-8-olate;
Sodium 3,3-dimethyl-7,8-dioxo-7,8-dihydro-3H-benzo[f]chromen-10-olate
8-Hydroxy-3-methyl-3-phenyl-3H-benzo[f]chromene-7,10-dione, and
8-Hydroxy-3,3-diphenyl-3H-benzo[f]chromene-7,10-dione.

4. A method uocording to claim 1 wherein the compound of formula (1) is selected from the group consisting of:
8-hydroxy-3,3-dimethyl-3H-naphtho[2,1-b]pyran-7,10-dione,
8-hydroxy-3,3-dimethyl-1,2-dihydro-3H-naphtho[2,1-b]pyran-7,10-dione).

5. The method of claim 1 wherein the compound of formula (1) is a compound of formula (3): 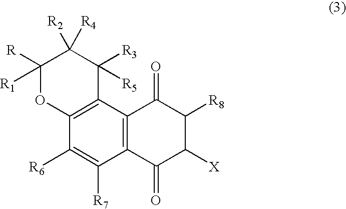
wherein X is OH, OR₉ or halo
R and R₁ are independently selected from H, C₁₋₆ alkyl, C₂₋₆ alkenyl, C₂₋₆ alkynyl, C₃₋₆ cycloalkyl, aryl, or together with the carbon atom to which they are attached form a saturated or unsaturated C₃₋₆ carbocyclic ring;
R₂ and R₃ are independently selected from H, C₁₋₆ alkyl, C₂₋₆ alkenyl, C₂₋₆ alkynyl, C₃₋₆ cycloalkyl or together with the bond between the carbon atoms to which they are attached form a double bond;
R₄ is selected from H, C₁₋₆ alkyl, C₂₋₆ alkenyl, C₂₋₆ alkynyl, C₃₋₆ cycloalkyl, halo or NR₁₀R₁₀ or together with R₅ and the bond between the carbon atoms to which R₄ and R₅ are attached, form a double bond;
R₅ is selected from H, C₁₋₆ alkyl, C₂₋₆ alkenyl, C₂₋₆ alkynyl, C₃₋₆ cycloalkyl, OH, OR₉, halo or NR₁₀R₁₀ or together with R₄ and the bond between the carbon atoms to which R₄ and R₅ are attached, form a double bond;
R₆ and R₇ are independently selected from H, C₁₋₆ alkyl, C₂₋₆ alkenyl, C₂₋₆ alkynyl, C₃₋₆ cycloalkyl, OH or OR₉;
R₈ is independently selected from H, C₁₋₆ alkyl, C₂₋₆ alkenyl, C₂₋₆ alkynyl, C₃₋₆ cycloalkyl, OH, OR₉ or halo; R₉ is C₁₋₆ alkyl, C₂₋₆ alkenyl, C₂₋₆ alkynyl, C₃₋₆ cycloalkyl, aryl, C(═O)R₁₁ or S(O)₂R₁₂ or OR₉ is an amino acid residue;
each R₁₀ is independently selected from H and C₁₋₆ alkyl;
R₁₁ is C₁₋₂₁ alkyl, C₂₋₂₁ alkenyl, C₂₋₂₁ alkynyl, C₃₋₆ cycloalkyl, C₃₋₆ cycloalkyl C₁₋₆ alkyl, aryl or aryl C₁₋₆ alkyl; and
R₁₂ is C₁₋₆ alkyl, C₂₋₆ alkenyl, C₂₋₆ alkynyl or aryl.

6. A method according to any one of claims 1 to 5 further comprising administering a second therapeutic agent.

7. A compound of Formula (1) or a pharmaceutically acceptable derivative, salt or prodrug thereof: 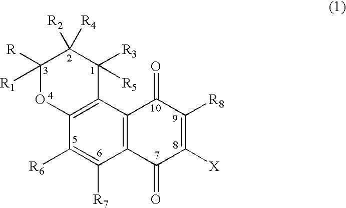
wherein X is OH, OR₉ or halo;
R and R₁ are independently selected from H, C₁₋₆ alkyl, C₂₋₆ alkenyl, C₂₋₆ alkynyl, C₃₋₆ cycloalkyl, aryl, or together with the carbon atom to which they are attached form a saturated or unsaturated C₃₋₆ carbocyclic ring;
R₂ and R₃ are independently selected from H, C₁₋₆ alkyl, C₂₋₆ alkenyl, C₂₋₆ alkynyl, C₃₋₆ cycloalkyl or together with the bond between the carbon atoms to which they are attached form a double bond;
R₄ und R₅ are independently selected from H, C₁₋₆ alkyl, C₂₋₆ alkenyl, C₂₋₆ alkynyl, C₃₋₆ cycloalkyl, OH, OR₉, halo or NR₁₀R₁₀ or together with the bond between the carbon atoms to which they are attached form a double bond; R₉ is C₁₋₆ alkyl, C₂₋₆ alkenyl, C₂₋₆ alkynyl, C₃₋₆ cycloalkyl, aryl, C(═O)R₁₁ or S(O)₂R₁₂ or OR₉ is an amino acid residue;
each R₁₀ is independently selected from H and C₁₋₆ alkyl; R₁₁ is C₁₋₂₁ alkyl, C₂₋₂₁ alkenyl, C₂₋₂₁ akynyl, C₃₋₆ cycloalkyl, C₃₋₆ cycloalkyl C₁₋₆ alkyl, aryl or aryl C₁₋₆ alkyl; and R₁₂ is C₁₋₆ alkyl, C₂₋₆ alkenyl, C₂₋₆ alkynyl or aryl;
with the proviso that when R and R₁ are both methyl and R is OH or OR₉, R₅ is not selected from OH, OR₉ or NHR9.

8. A compound according to claim 7 wherein the compound of Formula (1) is a compound of formula (2); 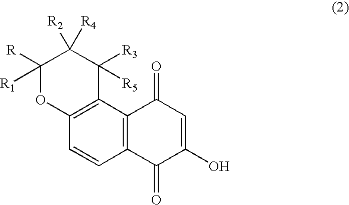
R and R₁ are independendy selected from H, C₁₋₆ alkyl, C₂₋₆ alkenyl, C₂₋₆ alkynyl, C₃₋₆ cycloalkyl, aryl, or together with the carbon atom to which they are attached form a saturated or unsaturated C₃₋₆ carbocycyclic ring; R₂ and R₃ are independently selected from H, C₁₋₆ alkyl, C₂₋₆ alkenyl, C₂₋₆ alkynyl, C₃₋₆ cycloalkyl or together with the bond between the carbon atoms to which they are attached form a double bond;
R₄ and R₅ are independently selected from H, C₁₋₆ alkyl, C₂₋₆ alkenyl, C₂₋₆ alkynyl, C₃₋₆ cycloalkyl, OH, OR₉, halo or NR₁₀R₁₀ or together with the bond between the carbon atoms to which they are attached form a double bond;
R₆ and R₇ are independently selected from H, C₁₋₆ alkyl, C₂₋₆ alkenyl, C₂₋₆ alkynyl, C₃₋₆ cycloalkyl, OH or OR₉;
R₈ is independently selected from H, C₁₋₆ alkyl, C₂₋₆ alkenyl, C₂₋₆ alkynyl, C₃₋₆ cycloalkyl, OH, OR₉ or halo;
R₉ is C₁₋₆ alkyl, C₂₋₆ alkenyl, C₂₋₆ alkynyl, C₃₋₆ cycloalkyl, aryl, C(═O)R₁₁ or S(O)₂R₁₂ or OR₉ is an amino acid residue;
each R₁₀ is independently selected from H and C₁₋₆ alkyl;
R₁₁ is C₁₋₂₁ alkyl, C₂₋₂₁ alkenyl, C₂₋₂₁ alkynyl, C₃₋₆ cycloalkyl, C₃₋₆ cycloalkyl C₁₋₆ alkyl, aryl or aryl C₁₋₆ alkyl; and
R₁₂ is C₁₋₆ alkyl, C₂₋₆ alkenyl, C₂₋₆ alkynyl or aryl.

9. A compound according to claim 7 wherein the compound of formula (1) is selected from the group consisting of:
8-hydroxy-3,3-dimethyl-3H-naphtho[2,1-b]pyran-7,10-dione,
8-hydroxy-3,3-dimethyl-1,2-dihydro-3H-naphtho[2,1-b]pyran-7,10-dione,
9-bromo-8-hydroxy-3,3-dimethyl-1,2-dihydro-3H-naphtho-[2,1-b]pyran-7,10-dione,
9-bromo-8-hydroxy-3,3-dimethyl-3H-naphtho[2,1-b]pyran-7,10-dione,
9-bromo-3,3-dimethyl-8-(4-methylbenzenesulfonyloxy)-1,2-dihydro-3H-naphtho[2,1-b]pyran-7,10-dione,
9-bromo-3,3-dimethyl-8-(4methylbenenesulfonyloxy)-3H-naphtho[2,1-b]pyran-7,10-dione,
8-acetoxy-3,3-dimethyl-3H-naphtho[2,1-b]pyran-7,10-dione,
2,9-dibromo-1,8-dihydroxy-3,3-dimethyl-1,2-dihydro-3H-naphtho[2,1-b]pyran-7,10-dione,
8,9-dichloro-3,3-dimethyl-1,2-dihydro-3H-naphtho[2,1-b]pyran-7,10-dione,
7,8,10-triacetoxy-3,3-dimethyl-3H-naphtho[2,1-b]pyran,
9-Bromo-8-hydroxy-3,3-dimethyl-3H-naphtho[2,1-b]pyran-7,10-dione.
9-Bromo-8-hydroxy-3,3-dimethyl-3H-naphtho[2,1-b]pyran-7,10-dione,
9-Bromo-3,3-dimethyl-8-(4-methylbenzenesulfonyloxy)-1,2-dihydro-3H-naphtho[2,1-b]pyran-7,10-dione.
9-Bromo-3,3-dimethyl-8-(4-methylbenzenesulfonyloxy)-3H-naphtho[2,1-b]pyran-7,10-dione,
8-Bromo-3,3-dimethyl-9-(4-methylbenzenesulfonyloxy)-3H-naphtho[2,1-b]pyran-7,10-dione,
8-Bromo-3,3-dimethyl-9-(4-methylbenzenesulfonyloxy)-1,2-dihydro-3H-naphtho[2,1-b]pyran-7,10-dione,
8,9-Dichloro-3,3-dimethyl -1,2-dihydro-3H-naphtho[2,1-b]pyran-7,10-dione,
Sodium 3,3-dimethyl-7,10-dioxo-7,10-dihydro-3H-benzo[f]chromen-8-olate;
Sodium 3,3-dimethyl-7,8-dioxo-7,8-dihydro-3H-benzo[f]chromen-10-olate;
8-Hydroxy-3-methyl-3-phenyl-3H-benzo[f]chromene-10-dione, and
8-Hydroxy-3,3-diphenyl-3H-benzo[f]chromene-7,10-dione,

10. A compound according to claim 7 wherein the compound of formula (1) is selected from the group consisting of:
8-hydroxy-3,3-dimethyl-3H-naphtho[2,1-b]pyran-7,10-dione,
8-hydroxy-3,3-dimethyl-1,2-dihydro-3H-naphtho[2,1-b]pyran-7,10-dione).

11. The compound of claim 7 wherein the compound of formula (1) is a compound of formula (3): 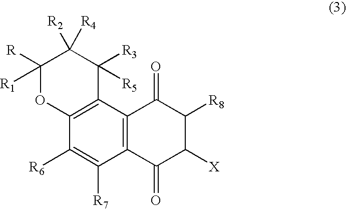
wherein X is OH, OR₉ or halo
R and R₁ are independently selected from H, C₁₋₆ alkyl, C₂₋₆ alkenyl, C₂₋₆ alkynyl, C₃₋₆ cycloalkyl, aryl, or together with the carbon atom to which they are attached form a saturated or unsaturated C₃₋₆ carbocyclic ring;
R₂ and R₃ are independently selected from H, C₁₋₆ alkyl, C₂₋₆ alkenyl, C₂₋₆ alkynyl, C₃₋₆ cycloalkyl or together with the bond between the carbon atoms to which they are attached form a double bond;
R₄ is selected from H, C₁₋₆ alkyl, C₂₋₆ alkenyl, C₂₋₆ alkynyl, C₃₋₆ cycloalkyl, halo or NR₁₀R₁₀ or together with R₅ and the bond between the carbon atoms to which R₄ and R₅ are attached, form a double bond;
R₅ is selected from H, C₁₋₆ alkyl, C₂₋₆ alkenyl, C₂₋₆ alkynyl, C₃₋₆ cycloalkyl, OH, OR₉, halo or NR₁₀R₁₀ or together with R₄ and the bond between the carbon atoms to which R₄ and R₅ are attached, form a double bond;
R₆ and R₇ are independently selected from H, C₁₋₆ alkyl, C₂₋₆ alkenyl, C₂₋₆ alkynyl, C₃₋₆ cycloalkyl, OH or OR₉;
R₈ is independently selected from H, C₁₋₆ alkyl, C₂₋₆ alkenyl, C₂₋₆ alkynyl, C₃₋₆ cycloalkyl, OH, OR₉ or halo;
R₉ is C₁₋₆ alkyl, C₂₋₆ alkenyl, C₂₋₆ alkynyl, C₃₋₆ cycloalkyl, aryl, C(═O)R₁₁ or S(O)₂R₁₂ or OR₉ is an amino acid residue;
each R₁₀ is independently selected from H and C₁₋₆ alkyl;
R₁₁ is C₁₋₂₁ alkyl, C₂₋₂₁ alkenyl, C₂₋₂₁ alkynyl, C₃₋₆ cycloalkyl, C₃₋₆ cycloalkyl C₁₋₆ alkyl, aryl or aryl C₁₋₆ alkyl; and
R₁₂ is C₁₋₆ alkyl, C₂₋₆ alkenyl, C₂₋₆ alkynyl or aryl.

12. A pharmaceutical composition comprising a compound according to any one of claims 7 to 11 and a pharmaccutieally acceptable carrier, diluent or excipient.
